(19) United States
(12) Patent Application Publication
(10) Pub. No.: US 2023/0124390 A1
(43) Pub. Date: Apr. 20, 2023

(54) POLYMER MATERIAL, NANOPARTICLE AND DRUG PREPARED THEREFROM, AND PREPARATION METHOD OF NANOPARTICLE

(71) Applicants: SOUTH CHINA UNIVERSITY OF TECHNOLOGY, Guangdong (CN); SUN YAT-SEN MEMORIAL HOSPITAL, SUN YAT-SEN UNIVERSITY, Guangdong (CN)

(72) Inventors: Menghua XIONG, Guangdong (CN); Yan BAO, Guangdong (CN); Liangqi HUANG, Guangdong (CN); Xiaochuan WANG, Guangdong (CN); Mingdong LIU, Guangdong (CN); Yangbin LIANG, Guangdong (CN); Jun WANG, Guangdong (CN)

(73) Assignees: SOUTH CHINA UNIVERSITY OF TECHNOLOGY, Guangdong (CN); SUN YAT-SEN MEMORIAL HOSPITAL, SUN YAT-SEN UNIVERSITY, Guangdong (CN)

(21) Appl. No.: 18/079,891

(22) Filed: Dec. 13, 2022

Related U.S. Application Data
(63) Continuation of application No. PCT/CN2021/102758, filed on Jun. 28, 2021.

(30) Foreign Application Priority Data
Jun. 30, 2020 (CN) 202010611437.4
Dec. 31, 2020 (CN) 202011633559.X

(51) Int. Cl. C08L 41/00; A61K 31/795; A61K 9/51; A61P 31/04; A61P 35/00

(52) U.S. Cl. CPC C08L 41/00; A61K 31/795; A61K 9/5192; A61P 31/04; A61P 35/00; B82Y 5/00

(57) ABSTRACT

The present disclosure provides a pH-sensitive membranolytic polymer material and a preparation method and application thereof. The pH-sensitive membranolytic polymer material has the structure shown in Formula (I). At normal physiological pH, the polymer material is hydrophobic neutral, and can be self-assembled into PEG coated nanoparticles with weak interaction with cell membrane; when the pH decreases, the polymer material can be protonated to form an amphiphilic structure consisting of hydrophobic domain and cationic domain, which has strong interaction with the cell membrane and strong membranolytic activity, so the polymer material can kill tumor cells or bacteria efficiently and selectively.

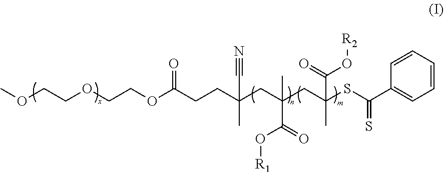

### CROSS-REFERENCE TO RELATED APPLICATION

This application is a continuation of international application of PCT application serial no. PCT/CN2021/102758 filed on Jun. 28, 2021, which claims the priority benefits of China application serial no. 202010611437.4, filed on Jun. 30, 2020 and China application serial no. 202011633559.X, filed on Dec. 31, 2020. The entirety of each of the above-mentioned patent application is hereby incorporated by reference herein and made a part of this specification.

### TECHNICAL FIELD

The present disclosure relates to the technical field of polymer materials and drugs, in particular to pH-sensitive membranolytic polymer materials and preparation method and applications thereof.

### DESCRIPTION OF RELATED ART

Some polymers can kill tumor cells, bacteria and other pathogens by destroying cell membrane, which has advantages of broad-spectrum killing effects and not easy to develop drug resistance. They have broad application prospects in the treatment of tumors and infectious diseases. For example, the application of LTX-315 cationic polypeptide can cause massive necrosis of tumor cells when applied to tumor therapy through intratumoral injection. It has achieved good results in melanoma, head and neck cancer, lymphoma, and breast cancer in combination with drugs such as Iprimumab and Pemtuzumab. The related research is in phase I of clinical study (https://clinicaltrials.gov/). The study of LL-37 polypeptide in the treatment of melanoma by intratumoral injection is also in phase I of clinical study (https://clinicaltrials.gov/). Maganins and other antimicrobial peptides are used in clinical treatment of bacterial infectious diseases. However, because such drugs can not only destroy tumor cells and pathogens with high efficiency, but also have strong cytotoxicity to normal tissue cells, and polypeptide drugs are easy to be degraded by protease. Most drugs are stopped in clinical research and currently the clinical use of drugs is mostly local injection therapy.

Some researchers have developed membranolytic polymers, such as polymethacrylate and polypeptide, by using positive primary amine or quaternary amine salt monomer and hydrophobic monomer. This kind of polymers have advantages, such as simple synthesis process and low cost, but it is still necessary to optimize the materials with high membranolytic activity and low toxicity to normal tissues and cells for promoting its clinical application. At present, researchers adjust the amphiphilic balance of membranolytic polymers by optimizing the types of cationic groups and hydrophobic groups, their proportions or spatial distribution. However, much work is still needed to optimize membranolytic polymers to meet clinical needs.

The interaction between polymer materials and cell membrane mainly depends on electrostatic and hydrophobic interactions. First, cationic domain is combined to the negatively charged cell membrane through electrostatic interaction. Then its hydrophobic domain is inserted into the lipid layer of cell membrane, which forms irreparable damage on the membrane, and thereby kills the cells. Generally speaking, simple cationic polymers are easily combined with cell membrane, but not easily inserted into cell membrane. While hydrophobic structural polymers are difficult to combine with cell membrane surface, which cannot effectively destroy cell membrane. Therefore, it is of great significance to design polymer materials that present cationic or hydrophobic structure in normal tissues, while transform into amphiphilic conformation composing of cationic domain and hydrophobic domain in diseased areas to solve the problem of high toxicity of membranolytic polymer materials.

A large number of studies have shown that tumor cells usually metabolize through glycolysis pathway to produce more lactic acid and carbon dioxide. However, because the lymphatic reflux in solid tumor tissue blocks the normal discharge of metabolic waste, the pH in peripheral environment of solid tumors is lower than that of normal body fluids, which usually stays between 6.4 to 7.0. The bacterial infection site also has acidic micro-environment, which is expected to be a response factor to the structural transformation of antibacterial polymers. Bacterial infection can lead to a local pH reduction through the production of organic acids (including lactic acid and acetic acid), and the acidity of inflammatory sites will be increased due to the production of lactic acid. It is of great significance to reasonably design the structure of polymer materials to obtain drugs with selective antibacterial activities in tumor and bacterial infection environments.

### SUMMARY OF THE DISCLOSURE

One of the objects of the present disclosure is to provide a kind of pH-sensitive polymer material, which is hydrophobic neutral under normal physiological pH, and can be self-assembled into PEG coated nanoparticles with weak interaction with cell membrane. When pH decreases, more tertiary amines are protonated to form an amphiphilic structure consisting of hydrophobic domain and cationic domain, which has strong interaction with cell membrane and strong membranolytic activity, so that it can kill tumor cells or bacteria with high efficiency and selectivity.

The specific technical solutions are as follows:

A polymer material, having the structure shown in Formula (I):

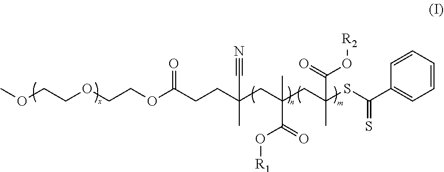

wherein R₁ is selected from —R₃—N(R₄R₅);
R₂ is selected from: alkyl, aryl, aryl substituted alkyl;
R₃ is selected from: alkylene;
R₄ and R₅ are independently selected from alkyl groups, or R₄ and R₅ together with the attached nitrogen atom form a heterocyclic alkyl group;
x is greater than 0;
n+m is not less than 20.
On the other hand, the present disclosure also provides a preparation method of pH-sensitive polymer materials described above.

The specific technical solutions are as follows:

A preparation method of pH-sensitive polymer material, including the following steps:

mPEG-CPDB, monomer 1, monomer 2 and initiator are dissolved in organic solvent and react in a closed manner under the protection of nitrogen or inert gas;

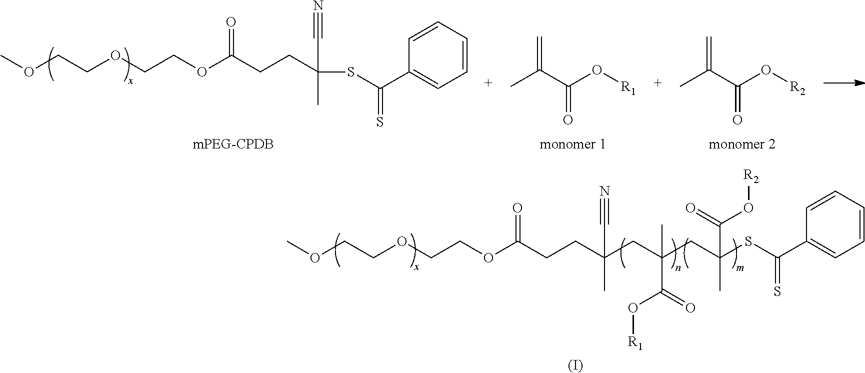

On the other hand, the present disclosure also provides nanoparticles of pH-sensitive polymer material.

The Nanoparticles of pH-sensitive polymer material, is formed by self-assembly in water medium of the above-identified pH-sensitive polymer material.

On the other hand, the present disclosure also provides a preparation method of the pH-sensitive polymer material nanoparticles.

A preparation method of nanoparticles of pH-sensitive polymer material, comprising the following steps: dissolving the pH-sensitive polymer material in dimethylformamide, adding the obtained solution into deionized water by drops, continuing stirring, and removing the solvent through dialysis to obtain the nanoparticles of pH-sensitive polymer material.

On the other hand, the present disclosure also provides applications of the pH-sensitive polymer material.

An application of the pH-sensitive polymer material in the prevention and/or treatment of tumors.

An application of the pH-sensitive polymer material in the treatment of bacterial infection.

On the other hand, the present disclosure also provides a drug for preventing and/or treating tumors.

The drug for preventing and/or treating tumors, is prepared from active ingredients and pharmaceutically acceptable excipients, wherein the active ingredients comprise the above-identified pH-sensitive polymer material.

On the other hand, the present disclosure also provides a drug for treating bacterial infections.

The drug for treating bacterial infections, is prepared from active ingredients and pharmaceutically acceptable excipients, wherein the active ingredients comprise the above-identified pH-sensitive polymer material.

The present disclosure provides a pH-sensitive polymer material, being composed of a hydrophilic polyethylene glycol, a tertiary amine polymethacrylate with a side group which has transformable hydrophilic and hydrophobic properties, and a polymethacrylate with a side hydrophobic group. At normal physiological pH, the tertiary amines in the polymer 5aterial are hydrophobic and electrically neutral, and the polymethacrylate segment is in a hydrophobic conformation. In addition, the polymer material can self-assemble into nanoparticles with PEG as shell, which has weak interaction with cell membrane, therefore it has the advantage of less toxicity to normal tissues during internal circulation, and furthermore, the PEG shell can improve the bio-compatibility of the polymer material and prolongs its blood circulation time; under the slightly acidic pH condition of tumor tissue or bacterial infection site, the tertiary amine part of the polymer material will be protonated, and consequently the polymethacrylate fragment will form an amphiphilic structure consisting of hydrophobic domain and cationic domain, therefore it has strong interaction with cell membrane and strong membranolytic activity, and thus it can efficiently and selectively kill tumor cells or bacteria. The polymer material of the present disclosure can be used to prepare drugs for anti-tumor or treatment for bacterial infection, which have the advantages of good anti-tumor and antibacterial effects, high selectivity and low toxicity.

### BRIEF DESCRIPTION OF DRAWINGS

FIG. 1 is the nuclear magnetic resonance (NMR) spectrum of macromolecular chain transfer agent mPEG-CPDB.

FIG. 2 is the NMR spectrum of methacrylate monomer C5-MA.

FIG. 3 is the NMR spectrum of methacrylate monomer C6-MA.

FIG. 4 is the NMR spectrum of methacrylate monomer C7-MA.

FIG. 5 is the NMR spectrum of methacrylate monomer DI-MA.

FIG. 6 is the NMR spectra of polymer materials P (C6-Me_{y})₈₀, P(C6-E_{y})₈₀ and P (C6-Bu_{y})₈₀ (y=10, 20, 30) prepared in Embodiment 1.

FIG. 7 is the NMR spectra of polymer materials P(C6-H_{y})₈₀, P(C6-IO_{y})₈₀ and P(C6-T_{y})₈₀ (y=5, 10, 20) prepared in Embodiment 1.

FIG. 8 is the NMR spectra of polymer materials P(C6-Ph₂₀)₈₀ and P(C6-NPh₂₀)₈₀ prepared in Embodiment 1.

FIG. 9 is NMR spectra of polymer materials P(C6-Bn_{y})₈₀(y=5, 10, 20, 30, 40) prepared in Embodiment 1.

FIG. 10 shows the variation of the protonation rate of polymer material prepared in Embodiment 1 and Embodiment 2 with pH.

FIG. 11 shows the cytotoxicity (a and b) of the polymer material prepared in Embodiment 1 at pH 7.4 and the maximum lethal dose (c) in mice.

FIG. 12 shows the cytotoxicity of the polymer material prepared in Embodiment 1 at pH6.8.

FIG. 13 is the NMR spectra of the polymer materials P(C5-Bn_{y})₈₀ (y=20, 30, 35, 40)prepared in Embodiment 2.

FIG. 14 is the NMR spectra of the polymer materials P(C7-Bn_{y})₈₀ (y=20, 30, 40) prepared in Embodiment 2.

FIG. 15 is the NMR spectra of the polymer materials P(DE-Bn_{y})₈₀ (y=20, 30, 35, 40) prepared in Embodiment 2.

FIG. 16 shows the cytotoxicity of the polymer material prepared in Embodiment 2 at pH7.4 and pH-6.8.

FIG. 17 is the NMR spectra of polymer materials with different polymerization degrees prepared in Embodiment 3.

FIG. 18 shows the cytotoxicity of polymer materials with different polymerization degrees prepared in Embodiment 3 at pH-6.8.

FIG. 19 is the NMR spectra of the polymer materials with different molecular weights of mPEG prepared in Embodiment 3.

FIG. 20 shows the cytotoxicity of the polymer material with different molecular weights of mPEG prepared in Embodiment 3 at pH 7.4 and pH 6.8.

FIG. 21 shows the potential (a), particle size (b), morphology (c) and NMR spectra (d) of P (C6-Bn₂₀)₈₀ nanoparticles at different pH values.

FIG. 22 shows the cytotoxicity of (C6-Bn₂₀)₈₀ nanoparticles to different tumor cells at different pH values.

FIG. 23 shows the effects of different temperatures and different endocytosis inhibitors on the cytotoxicity of P (C6-Bn₂₀)₈₀ nanoparticles at different pH values.

FIG. 24 is the transmission electron microscope picture of Panc-02 cells co-incubated with P (C6-Bn₂₀)₈₀ polymer material at different pH values.

FIG. 25 is the scanning electron microscope of the Panc-02 cells after being treated with P (C6-Bn₂₀)₈₀ polymer material.

FIG. 26 shows the anti-tumor effect of P (C6-Bn₂₀)₈₀ polymer material on Panc02 cell model in vivo.

FIG. 27 shows the anti-tumor effect of P(C6-Bn₂₀)₈₀ polymer material on B16-F10 cells, CT-26 cells and A549 cell models in vivo.

FIG. 28 shows the results of bactericidal activity of P(C6-Bn₂₀)₈₀ and P (C7-Bn₂₀)₈₀, wherein a is the killing effect of polymer materials on Escherichia coli at pH 5.00, 5.50, 6.00 and 7.40; b-f are the killing effects of the polymer material on Salmonella, Staphylococcus aureus, Klebsiella pneumoniae, Pseudomonas aeruginosa and Enterococcus faecalis at pH 6.00.

FIG. 29 shows the surface morphology of Escherichia coli observed by scanning electron microscope (SEM) after being incubated with P (C6-Bn₂₀)₈₀ and P (C7-Bn₂₀)₈₀ at pH 7.40 and 6.00.

### DESCRIPTION OF EMBODIMENTS

In the following embodiments of the present disclosure, the experimental methods without specifying specific conditions usually follow conventional conditions, or the specific conditions as recommended by the manufacturer. The various common chemical reagents used in the embodiments are commercially available products.

Unless otherwise defined, all technical and scientific terms used in the present disclosure are the same as commonly understood by those skilled in the art of the present disclosure. Terms used in the description of the present disclosure are only for the purpose of describing specific embodiments, but do not limit the present disclosure.

The terms “including”, “having”, and any variation thereof in the present disclosure are intended to cover the non-exclusive inclusions. For example, processes, methods, devices, products, or equipment that include a series of steps are not limited to the steps or modules that are already listed, but selectively includes steps that are not listed, or includes other steps inherent to these processes, methods, products, or equipment.

In the present disclosure, “multiple” refers to two or more. “And/or” describes the association relationship of the associated objects, indicating that there can be three types of relationships. For example, “A and/or B” indicates three situations: A exists alone, A and B exist at the same time, and B exists alone. The character “/” generally indicates that the context objects are in an “or” relationship.

The present disclosure provides a pH-sensitive polymer material with the structure shown in Formula (I) in some embodiments:

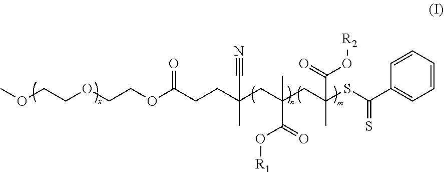

wherein R₁ is selected from —R₃—N(R₄R₅);
R₂ is selected from: alkyl, aryl, aryl substituted alkyl;

R₃ is selected from: alkylene;

R₄ and R₅ are independently selected from alkyl groups, or R₄ and R₅ together with the attached nitrogen atom form a heterocyclic alkyl group;
x is greater than 0;
n+m is not less than 20.

In some embodiments, R₂ is selected from: C₁-C₁₅ alkyl, C₆-C₁₄ aryl, C₆-C₁₄ aryl substituted C₁-C₁₅ alkyl.

In some embodiments, R₂ is selected from: C₁-C₁₂ alkyl, phenyl, naphthyl phenyl-substituted C₁-C₃ alkyl, naphthyl-substituted C₁-C₃ alkyl alkyl.

In some embodiments, R₂ is selected from: methyl, ethyl, propyl, isopropyl, butyl, pentyl, hexyl, heptyl, isooctyl, nonyl, decyl, undecyl, dodecyl, tetradecyl, phenyl, naphthyl, benzyl, naphthalene methyl.

In some embodiments, R₃ is selected from: C₁-C₆ alkylene.

In some embodiments, R₃ is selected from: C₁-C₃ alkylene.

In some embodiments, R₃ is selected from: methylene, ethylidene, propylidene.

In some embodiments, R₄ and R₅ are independently selected from C₁-C₆ alkyl, or R₄ and R₅ together with the attached nitrogen atom form a 5-10 membered heterocyclic alkyl.

In some embodiments, R₄ and R₅ are independently selected from C₁-C₄ alkyl, or R₄ and R₅, together with the attached nitrogen atom form a 5-8 membered heterocyclic alkyl.

In some embodiments, R₄ and R₅ together with the attached nitrogen atom form the following groups:

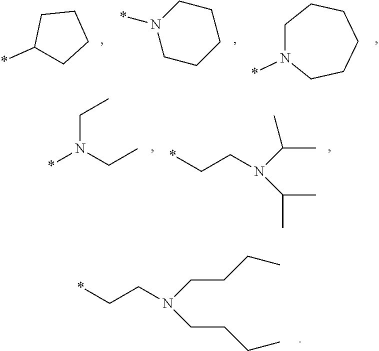

In some embodiments, the polymer material has the structure shown in Formula (II):

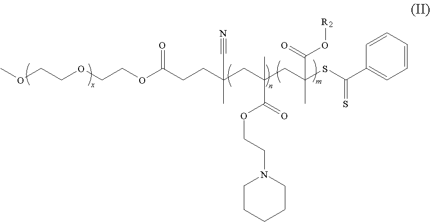

wherein R₂ is selected from: methyl, ethyl, propyl, isopropyl, butyl, pentyl, hexyl, heptyl, isooctyl, nonyl, decyl, undecyl, dodecyl, tetradecyl, phenyl, naphthyl, benzyl, naphthalene methyl.

In some embodiments, the polymer material has the structure shown in Formula (III):

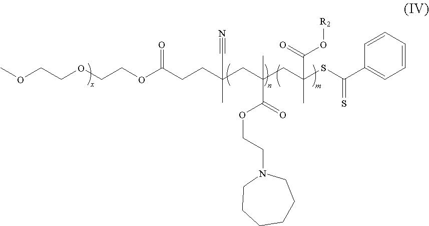

wherein R₂ is selected from: methyl, ethyl, propyl, isopropyl, butyl, pentyl, hexyl, heptyl, isooctyl, nonyl, decyl, undecyl, dodecyl, tetradecyl, phenyl, naphthyl, benzyl, naphthalene methyl.

In some embodiments, the pH-sensitive polymer material has the structure shown in Formula (IV):

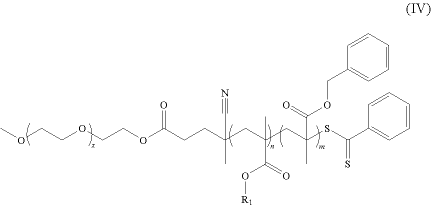

wherein R₁ is selected from:

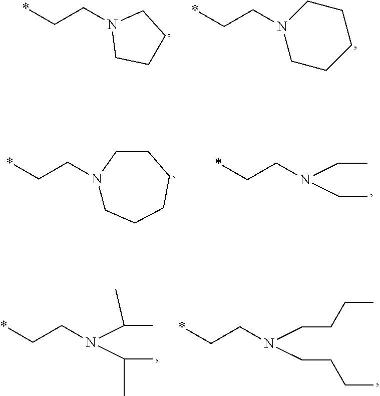

In some embodiments, n+m is not less than 30.

In some embodiments, n+m is not less than 40.

In some embodiments, n+m is not less than 50.

In some embodiments, n+m is not less than 60.

In some embodiments, n+m is not less than 70.

In some embodiments, n+m is 70-300 .

In some embodiments, n+m is 75-200.

In some embodiments, m is 5%-50% of n+m.

In some embodiments, m is 15%-40% of n+m.

In some embodiments, m is 18%-30% of n+m.

In some embodiments, m is 20%-25% of n+m.

In some embodiments, x is 10-250.

In some embodiments, the pH-sensitive polymer material has the structure shown in Formula (V) or Formula (VI):

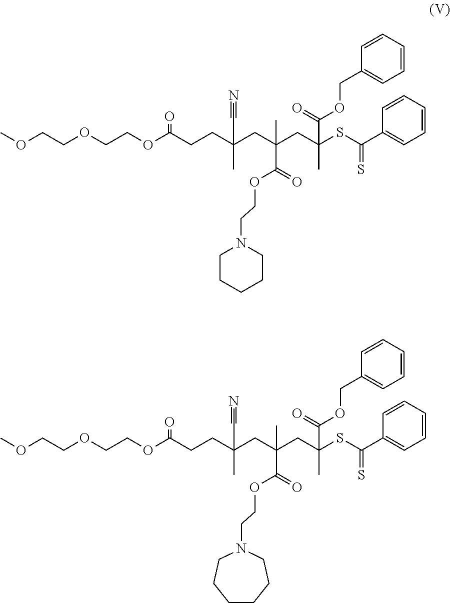

In the compounds of the present disclosure, when any variable (eg, R₂, R₃, etc.) occurs more than once in any component, its definition at each occurrence is independent of other definition at each occurrences. Likewise, combinations of substituents and variables are permissible as long as such combinations stabilize the compound. The line drawn into a ring system from a substituent indicates that the indicated bond may be attached to any substitutable ring atom. If the ring system is polycyclic, it means that such bonds are only attached to any suitable carbon atoms of the adjacent ring. It should be understood that an ordinary skilled in the art can select substituents and substitution patterns for the compounds of the present disclosure to provide compounds that are chemically stable and readily synthesized from the available starting materials through the techniques in the art and the methods described below. If the substituent itself is substituted by more than one group, it should be understood that these groups may be on the same carbon atom or on different carbon atoms as long as the structure is stabilized. The phrase “optionally substituted by one or more substituents” is considered equivalent to the phrase “optionally substituted by at least one substituent” , and in such case the preferred embodiment will have 0-3 substituents.

The term “alkyl” in the present disclosure is meant to include branched and straight chain of saturated aliphatic hydrocarbon groups having the specified number of carbon atoms. For example, the definition of “C₁-C₆” in “C₁-C₆ alkyl” includes groups having 1, 2, 3, 4, 5 . . . or 6 carbon atoms arranged in a straight or branched chain. For example, “C₁-C₆ alkyl” specifically includes methyl, ethyl, n-propyl, isopropyl, n-butyl, tert butyl, isobutyl, pentyl, and hexyl.

The term “heterocycloalkyl” refers to a saturated monocyclic cyclic substituent, in which one or more ring atoms are selected from the heteroatom of N, O or S (O) in (wherein, in is an integer of 0-2), and the remaining ring atoms are carbon, such as piperidyl, pyrrolyl, etc.

In some embodiments, the preparation method of pH-sensitive polymer material are applied, including the following steps:

mPEG-CPDB, monomer 1, monomer 2 and initiator are dissolved in organic solvent to react in a closed manner under the protection of nitrogen or inert gas;

the reaction formula is as follows:

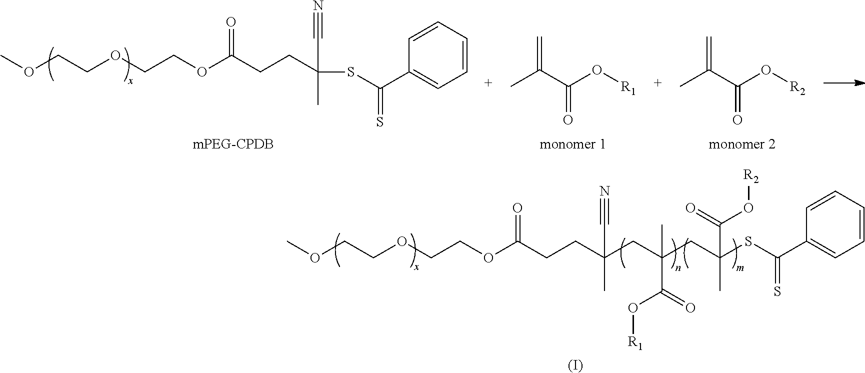

In some embodiments, the reaction time is 18 hours-24 hours, and the reaction temperature is 70° C.-80° C.

In some embodiments, the organic solvent is 1,4-dioxane.

In some embodiments, the initiator is azodiisobutyronitrile.

The present disclosure also provides nanoparticles of the pH-sensitive polymer material in some embodiments, which are formed by self-assembly of the above-identified pH-sensitive polymer material in the water medium.

In some embodiments, the present disclosure also provides a preparation method of the above-identified pH-sensitive polymer material nanoparticles, comprising the following steps: dissolving the polymer material in dimethylformamide, and then adding the obtained solution into deionized water by drops under stirring, continuing stirring, and removing the solvent through dialysis to obtain the pH-sensitive polymer material nanoparticles.

In some embodiments, the preparation method of the pH-sensitive polymer material nanoparticles comprises the following steps: dissolving the polymer material in dimethylformamide at a ratio of 45-55 mg: 1 mL, and then adding the obtained solution into deionized water by drops under stirring at a speed of 1200-1700 rpm; continuing to stir at a speed of 800-1200 rpm for 8 minutes-12 minutes, and removing the solvent by dialysis using a dialysis bag with a cutoff molecular of 10000˜20000 to obtain the pH-sensitive polymer material nanoparticles.

The present disclosure also provides an application of the pH-sensitive polymer material in prevention and/or treatment of tumors.

In some embodiments, the tumors are pancreatic cancer, melanoma, colorectal cancer, lung cancer, tongue squamous cell cancer, cervical cancer, ovarian cancer, osteosarcoma, liver cancer, breast cancer, bladder cancer, epithelial ovarian cancer.

The present disclosure also provides an application of the pH-sensitive polymer material in the treatment of bacterial infection.

In some embodiments, the bacteria are gram-negative bacteria, gram-negative pseudomonas, gram-positive staphylococcus, gram-positive coccus, gram-positive streptococcus.

In some embodiments, the bacteria are Escherichia coli, Salmonella, Staphylococcus aureus, Klebsiella pneumoniae, Pseudomonas aeruginosa, Enterococcus faecalis, Streptococcus pyogenes, Streptococcus pneumoniae, Acinetobacter baumannii, Diplococcus pneumoniae, Pseudomonas aeruginosa.

Some embodiments oaf the present disclosure also relate to drugs for preventing and/or treating tumors, which are prepared from active ingredients and pharmaceutically acceptable excipients, wherein the active ingredients comprise the pH-sensitive polymer material.

Some embodiments also relate to drugs for treating bacterial infections, which are prepared from active ingredients and pharmaceutically acceptable excipients, wherein the active ingredients comprise the pH-sensitive polymer material.

Some embodiments also relate to drugs for preventing and/or treating tumors, and treating bacterial infections, which can be used for non-human mammals or humans.

Some embodiments also relate to drugs for preventing and/or treating tumors, and treating bacterial infections, wherein the pharmaceutically acceptable excipients refer one or more compatible solid or liquid fillers or gel substances, which are suitable for human and must have sufficient purity and low toxicity.

“Compatibility” here refers to that each component in the composition can be mixed with the active ingredients of the present disclosure (pH-sensitive membranolytic polymer materials shown in Formula I-Formula VI), or intermingled between each other without significantly reducing the efficacy of the active ingredients.

In the present disclosure, the pharmaceutically acceptable excipients used in the medicine for preventing and/or treating tumors include but not limited to one or more of the following materials: solvent, excipients, fillers, compatibilizers, adhesives, humectants, disintegrating agents, retarders, absorption accelerators, adsorbents, diluents, solubilizers, emulsifiers, lubricants, wetting agents, suspending agents, flavoring agents, and spices.

Some embodiments of pharmaceutically acceptable excipients include cellulose and its derivatives (such as sodium carboxymethyl cellulose, sodium ethylcellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agent (such as sodium dodecyl sulfate), colorant, flavoring agent, stabilizer, antioxidant, preservative, pyrogen free water, etc.

There is no special restriction on application methods of the active ingredients or drug composition of the present disclosure, and typical application methods include (but not limited to) oral administration, rectal administration, parenteral administration (intravenous, intramuscular or subcutaneous), etc.

The solid dosage forms used for oral administration include capsules, tablets, pills, powders and granules.

In these solid dosage forms, the active ingredient is mixed with at least one conventional inert excipient (or carrier), such as sodium citrate or dicalcium phosphate, or with the following ingredients:

(a) fillers or compatibilizers, such as starch, lactose, sucrose, glucose, mannitol, and silicic acid;
(b) adhesives, such as hydroxymethyl cellulose, alginate, gelatin, polyvinylpyrrolidone, sucrose, and arabic gum;
(c) humectants, such as glycerin;
(d) disintegrating agents, such as agar, calcium carbonate, potato starch or cassava starch, algic acid, some composite silicates, and sodium carbonate;
(e) slow solvent, such as paraffin;
(f) absorption accelerators, such as quaternary amine compounds;
(g) wetting agents, such as cetyl alcohol and glyceryl monostearate;
(h) adsorbents, such as kaolin; and
(i) lubricants, such as talc, calcium stearate, magnesium stearate, solid polyethylene glycol, sodium dodecyl sulfate, or their mixtures.

In the case of capsules, tablets and pills, the dosage form may also include buffers.

The solid dosage form can also be prepared with coating and shell materials, such as casings and other materials known in the art. They may comprise an opaque agent. Furthermore, the release of active ingredients from such compositions may be released in a delayed manner in certain part of the digestive tract. Embodiments of coating and shell materials that can be used are polymers and waxes.

Liquid dosage forms for oral administration include pharmaceutically acceptable lotion, solutions, suspensions, syrups or tinctures. In addition to the active ingredients, the liquid dosage form may include inert diluents commonly used in the art, such as water or other solvents, solubilizers, emulsifiers (such as ethanol, isopropanol, ethyl carbonate, ethyl acetate, propylene glycol, 1,3-butanediol, dimethylformamide), and oil (especially cottonseed oil, peanut oil, corn germ oil, olive oil, castor oil and sesame oil or mixtures of these substances). In addition to these inert diluents, the composition may also include auxiliary agents, such as wetting agents, emulsifiers and suspending agents, sweeteners, flavoring agents and spices.

In addition to the active ingredients, the suspension may contain suspension agents, such as ethoxylated isooctadecanol, polyoxyethylene sorbitol and dehydrated sorbitol ester, microcrystalline cellulose, aluminum methoxide and agar, or mixtures of these substances.

Compositions for parenteral injection may include physiologically acceptable sterile aqueous or anhydrous solutions, dispersions, suspensions or emulsions, and sterile powders for re-dissolution into sterile injectable solutions or dispersions. Suitable aqueous and non-aqueous carriers, diluents, solvents or excipients include water, ethanol, polyols, and their suitable mixtures.

The following are specific embodiments.

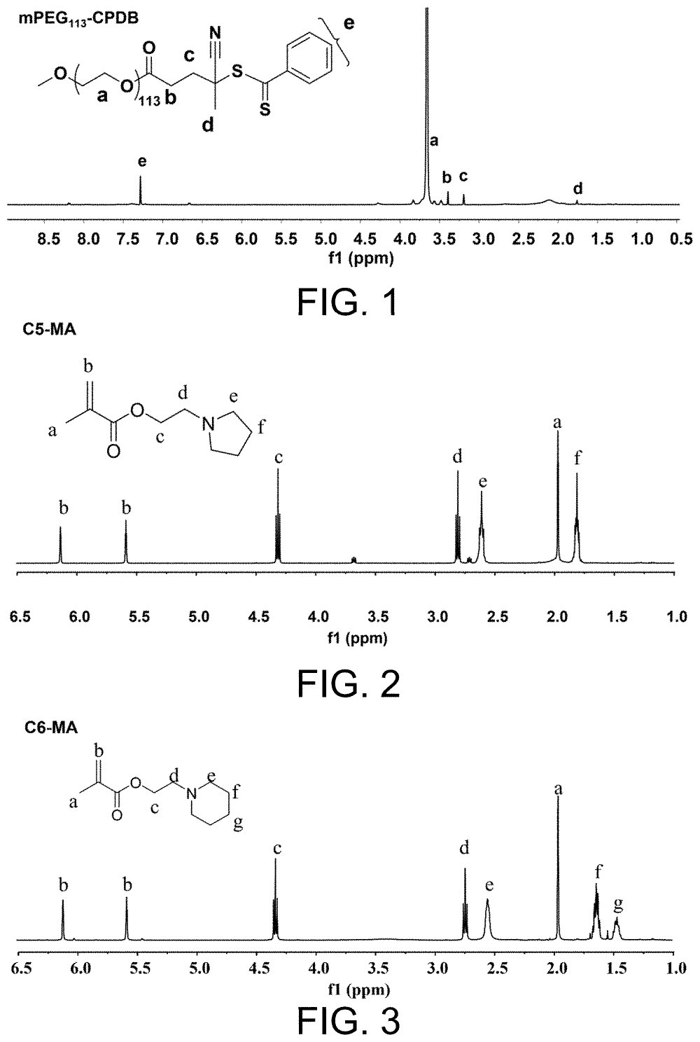

In the following embodiments, a series of membranolytic polymer materials shown in Formula (I) were synthesized by RAFT polymerization method. The reaction formula is as follows:

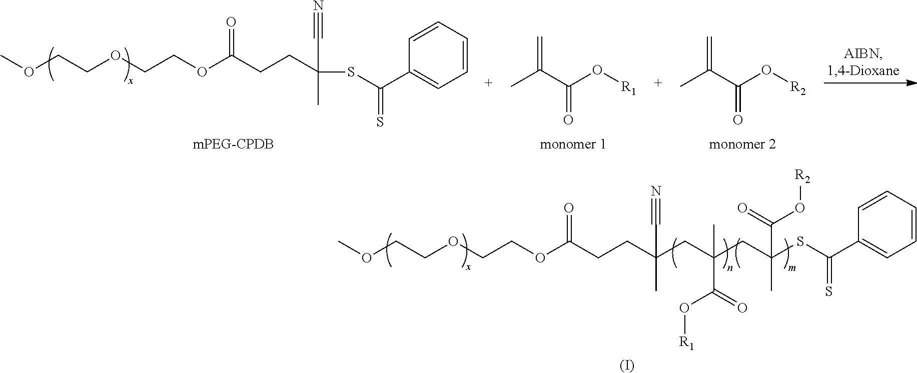

wherein mPEG-CPDB is a macro-chain mister agent; monomer 1 is a methacrylate with side group containing an ionizable tertiary amine, and monomer 2 is a methacrylate with side group containing hydrophobic groups.

Wherein, the macro-chain transfer agent, mPEG-CPDB, for polymerization was synthesized according to the preparation method described by Ma, X. P. et al. Ultra pH Sensitive Nanoprobe Library with Broad pH Tunability and Fluorescence Emissions. J. Am. Chem. Soc. 136. 11085-11092 (2014). Its NMR spectrum is shown in FIG. 1.

The methacrylate monomer with ionizable tertiary amine in the side group for polymerization can be purchased from the market. If the purchased monomer contains stabilizer 4-Methoxyphenol (MEHQ), it needs to be removed by distillation. Some commodities need to be synthesized by reaction of alcohol and acyl chloride if they are not available on the market. Specific synthetic reaction steps of monomers C5-MA, C6-MA, C7-MA, DI-MA are described by Li, H. J. et al. Smart Superstructures with Ultrahigh pH-Sensitivity for Targeting Acidic Tumor Microenvironment: Instantaneous Size Switching and Improved Tumor Penetration. ACS Nano. 10, 6753-6761 (2016).

The structural formulas of the synthesized CS-MA, C6-MA, C7-MA and DI-MA are as follows:

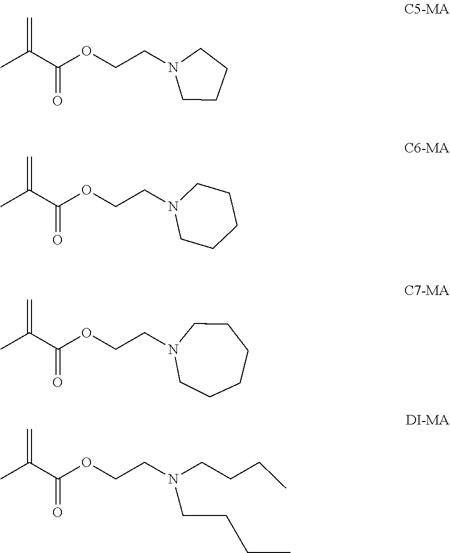 [SMILES: C=C(C)C(=O)OCCN1CCCC1 ; C=C(C)C(=O)OCCN1CCCCC1 ; C=C(C)C(=O)OCCN1CCCCCC1 ; C=C(C)C(=O)OCCN(CCCC)CCCC]

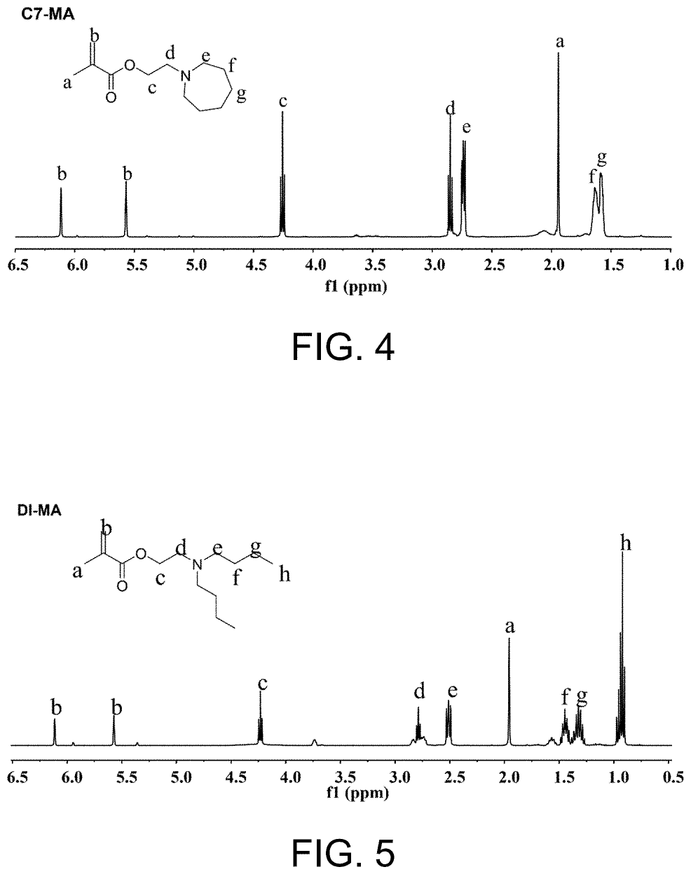

Their NMR spectra are shown in FIG. 2-FIG. 5.

Methacrylate monomer containing hydrophobic groups (hereinafter referred to as hydrophobic monomer) is directly purchased, but commercial products contain hydroquinone as a stabilizer, which has polymerization inhibition effect. Therefore, hydrophobic monomer used in reaction is obtained through atmospheric or vacuum distillation. Specific distillation temperature and pressure are in Table 1.

TABLE 1
Distillation conditions of methacrylate

| Monomer | Distillation temperature and pressure |
|---|---|
| Methyl methacrylate (MeMA) | 100° C./760 mmHg |
| Ethyl methacrylate (EMA) | 119° C./760 mmHg |
| Butyl methacrylate (BuMA) | 38~43° C./0.5 mmHg |
| Hexyl methacrylate (HMA) | 40~45° C./0.5 mmHg |
| Isooctyl methacrylate (IOMA) | 80~84° C./0.5 mmHg |
| Tetradecyl methacrylate (TMA) | 118~125° C./0.5 mmHg |
| Benzyl methacrylate (BnMA) | 95~100° C./0.5 mmHg |
| Phenyl methacrylate (PhMA) | 80~87° C./0.5 mmHg |

Embodiment 1:

### 1. Preparation and Characterization of Polymer Materials with R₁ being ethyl piperidine (C6) and R₂ being Different Hydrophobic Groups.

Polymer materials containing different types and proportions of hydrophobic groups with the tertiary amine being ethyl piperidine (C6) were prepared in this embodiment. These polymer materials were synthesized by the following RAFT polymerization method, and the reaction equation is as follows:

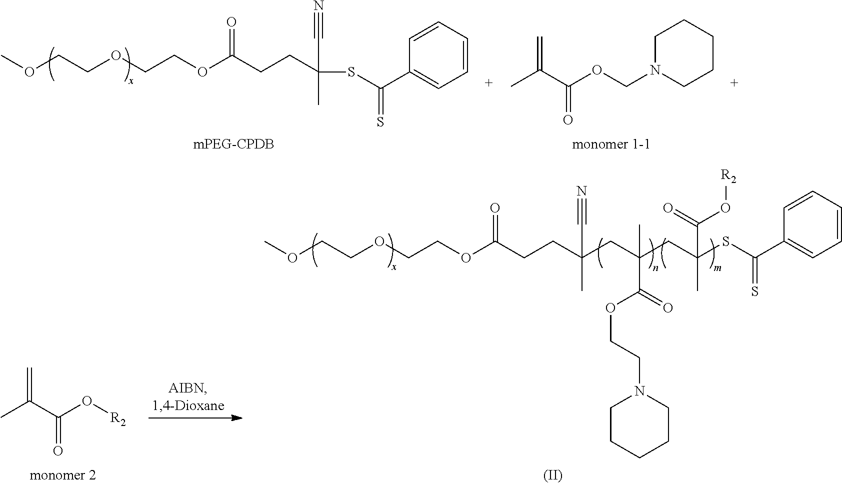

Wherein R₂ is methyl(MeMA),

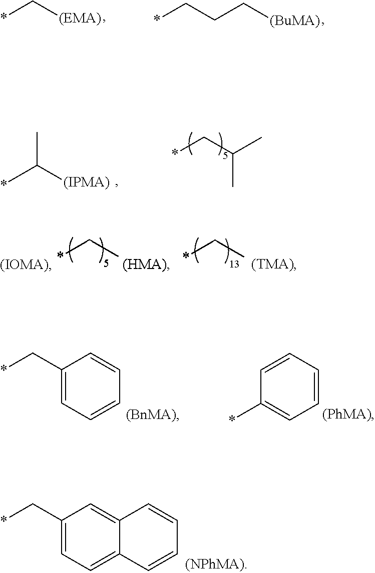

The specific steps are as follows:

0.2 g mPEG113-CPDB (wherein the molecular weight of PEG is 5000) was dissolved in 1,4-dioxane and placed in a reaction vessel, and then added C6-MA monomer and corresponding MeMA, EMA, BuMA, HMA, IOMA, TMA, BnMA, PhMA, NPhMA monomers according to the molar ratio shown in Table 2. After mixed evenly, added 2 mg of initiator azodiisobutyronitrile (AIBN) and used liquid nitrogen to freeze the mixture into solid; reduced the pressure, and the temperature was restored to room temperature to release oxygen from the reaction solution; then froze again. Repeated these steps for several times until there is no oxygen in the reaction vessel, then filled with high-purity nitrogen, sealed and reacted at 75° C. for 18-24 hours. After the reaction was completed, terminated the reaction by freezing in liquid nitrogen, and then the reaction solution was melted and dropped into n-hexane to obtain a corresponding orange-yellow polymer material.

The polymers prepared in this embodiment is abbreviated as P(R₁-R_{2(y)})_{z} (wherein P represents PEG, R₁ represents the type of methacrylate monomers with side group containing tertiary amine, R₂ represents the type of methacrylate monomers with hydrophobic groups, y represents the proportion of the hydrophobic monomer R₂ in the two monomers, and z represents the total polymerization degree of the two monomers) and the feeding ratios are shown in Table 2, wherein reactant R₁ refers to the methacrylate monomer 1 with side group containing tertiary amine (C6-MA in this embodiment), and reactant R₂ refers to the methacrylate monomer 2 containing hydrophobic groups.

TABLE 2
Feeding ratios of series polymer materials P(R₁ − R_{2(y)})_{z}

| | | Reactants | | |
|---|---|---|---|---|
| Abbreviation | | R₁ | R₂ | Feeding ratios |
| of polymer | Chain | (tertiary | (hydrophobic | (Chain transfer |
| materials | transfer agent | amine) | groups) | agent:R₁:R₂) |
| P(C6-Me₁₀)₈₀ | mPEG₁₁₃- | C6-MA | MeMA | 1:72:8 |
| P(C6-Me₂₀)₈₀ | CPDB | | MeMA | 1:64:16 |
| P(C6-Me₃₀)₈₀ | | | MeMA | 1:56:24 |
| P(C6-E₁₀)₈₀ | | | EMA | 1:72:8 |
| P(C6-E₂₀)₈₀ | | | EMA | 1:64:16 |
| P(C6-E₃₀)₈₀ | | | EMA | 1:56:24 |
| P(C6-Bu₁₀)₈₀ | | | BuMA | 1:72:8 |
| P(C6-Bu₂₀)₈₀ | | | BuMA | 1:64:16 |
| P(C6-Bu₃₀)₈₀ | | | BuMA | 1:56:24 |
| P(C6-H₅)₈₀ | | | HMA | 1:76:4 |
| P(C6-H₁₀)₈₀ | | | HMA | 1:72:8 |
| P(C6-H₂₀)₈₀ | | | HMA | 1:64:16 |
| P(C6-IO₅)₈₀ | | | IOMA | 1:76:4 |
| P(C6-IO₁₀)₈₀ | | | IOMA | 1:72:8 |
| P(C6-IO₂₀)₈₀ | | | IOMA | 1:64:16 |
| P(C6-T₅)₈₀ | | | TMA | 1:76:4 |
| P(C6-T₁₀)₈₀ | | | TMA | 1:72:8 |
| P(C6-T₂₀)₈₀ | | | TMA | 1:64:16 |
| P(C6-Bn₅)₈₀ | | | BnMA | 1:76:4 |
| P(C6-Bn₁₀)₈₀ | | | BnMA | 1:72:8 |
| P(C6-Bn₂₀)₈₀ | | | BnMA | 1:64:16 |
| P(C6-Bn₃₀)₈₀ | | | BnMA | 1:56:24 |
| P(C6-Bn₄₀)₈₀ | | | BnMA | 1:48:32 |
| P(C6-NPh₂₀)₈₀ | | | NPhMA | 1:64:16 |
| P(C6-Ph₂₀)₈₀ | | | PhMA | 1:64:16 |

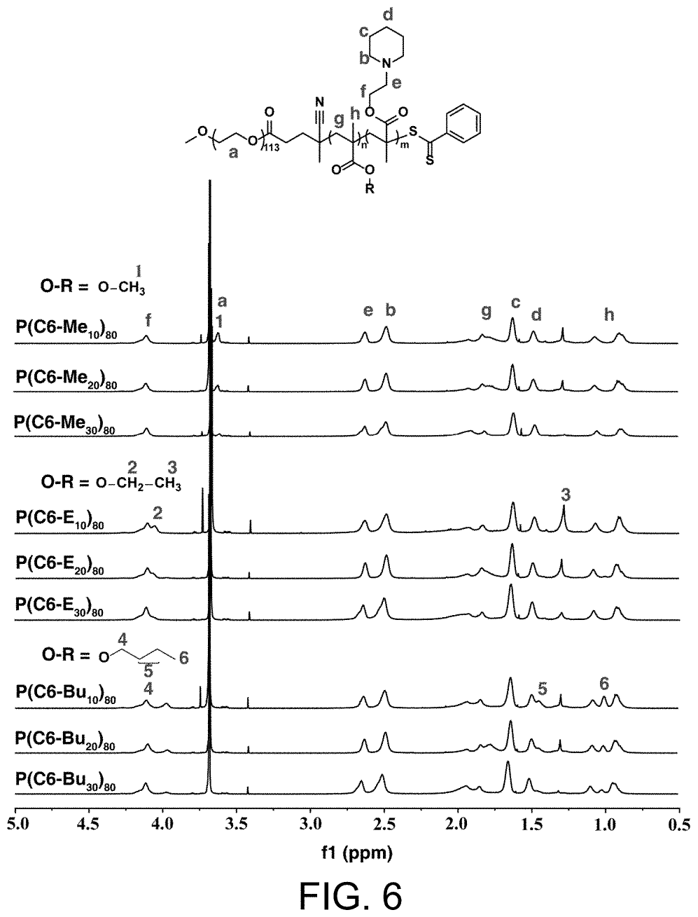

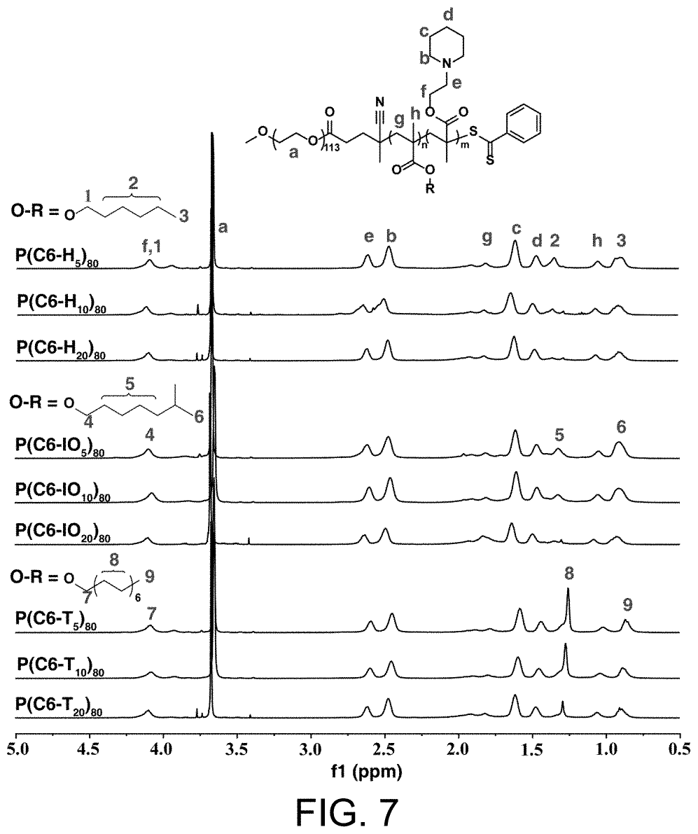

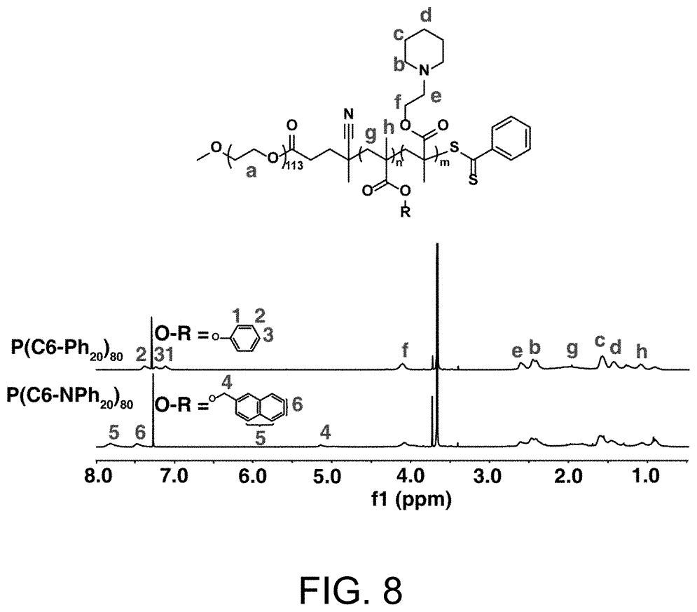

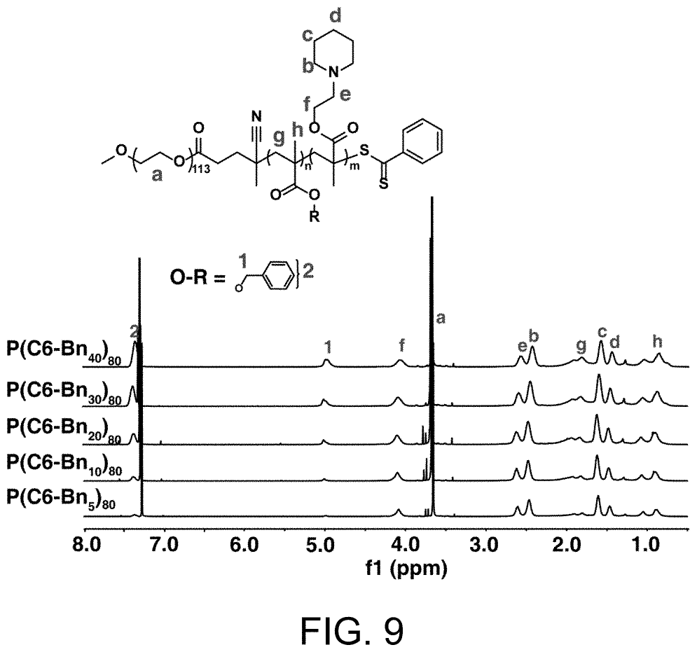

The NMR spectra of the obtained polymer materials are shown in FIG. 6 to FIG. 9. 3.66 ppm (a) is the proton signal peak of the hydrogen atoms on the polyethylene glycol chain (—CH₂CH₂O—), 1.85 ppm (g) is the proton signal peak of the hydrogen atoms on the polymer main chain (—CH₂—); 0.9-1.05 ppm (h) is the proton signal peak of the methyl hydrogen atoms (—CH₃) on the side chain; 2.48 ppm (b, —NCH₂—), 1.62 ppm (c, —CH₂—) and 1.47 ppm (d, —CH₂—) are the proton signal peaks of hydrogen atoms on the C6 heterocycle; 2.62 ppm (e, —CH₂N—) and 4.10 ppm (f, —OCH₂—) are the proton signal peaks of the hydrogen atoms on the side chain. For P(C6-Me_{y}), its characteristic peak at 3.60 ppm (1) is the proton signal peak of the hydrogen atoms of the methoxy group (—OCH₃) on the hydrophobic side chain; the characteristic peaks of P(C6-E_{y}) at 4.05 ppm (2, —OCH—) and 1.28 ppm (3, —CH₃) are the proton signal peaks of the hydrogen atoms of the ethyl group on the hydrophobic side chain; the characteristic peaks of P(C6-Bu_{y}) at 4.09 ppm (4, —OCH₂—), 1.42 ppm (5, —CH₂CH₂—) and 0.97 ppm (6, —CH₃) are the proton signal peaks of the hydrogen atoms of the butyl group on the hydrophobic side chain. The characteristic peaks of P(C6-H_{y}) at 4.09 ppm (1, —OCH₂—), 1.34 ppm [2, —(CH₂) 4-], and 0.88 ppm (3, —CH₃) are the proton signal peaks of the hydrogen atoms of the hexyl group on the hydrophobic side chain; the characteristic peaks of P(C6-IO_{y}) at 4.09 ppm (4, —OCH₂—), 1.31 ppm [5, —(CH₂)₄-], and 0.90 ppm (6, —CH₃) are the proton signal peaks of the hydrogen atoms of the isooctyl group on the hydrophobic side chain; the characteristic peaks of P(C6-T_{y}) at 4.09 ppm (7, —OCH₂—), 1.28 ppm [8, —CH₂)12-] and 0.89 ppm (9, —CH₃) are the proton signal peaks of the hydrogen atoms of the butyl group on the hydrophobic side chain. For P(C6-Bn_{y}), its characteristic peaks at 5.00 ppm (1, —OCH₂—), 7.34 ppm [2, (—CH═)5] are the proton signal peaks of the hydrogen atoms of the benzyl group on the hydrophobic side chain. For P(C6-Ph₂₀), its characteristic peaks at 7.11 ppm (1, —CH═), 7.38 ppm (2, —CH═) and 7.23 ppm (3, —CH═) are the proton signal peaks of the hydrogen atoms of the phenyl group on the hydrophobic side chain; and the characteristic peaks of P(C6-NPh₂₀) at 5.13 ppm (4, —OCH₂—), 7.82 ppm [5, (—CH═)₄], and 7.48 ppm [6, (—CH═)₂] are the proton signal peaks of the hydrogen atoms of the naphthalene group on the hydrophobic side chain.

The number average molecular weight (Mₙ), weight average molecular weight (M_{W}), and single polymer dispersity index (PDI) were calculated according to the nuclear magnetic integration and the positions of the proton signal peaks of hydrogen atoms, combined with the GPC results, and the molecular weight calculated by the integration of the hydrogen NMR spectra are listed in the following table. It is judged that the polymer materials designed in this embodiment were successfully synthesized, and their polymerization degrees were controlled at about 80 (see Table 3 for relevant data). The molecular weight (M_{w}, Mₙ) and the molecular weight distribution (PDI) of the superscripted with a in the header of the table were obtained by GPC test: the polymers were dissolved in tetrahydrofuran at a final concentration of 5-10 mg/mL, Detection was performed by gel permeation liquid chromatography equipped with a Waters 1515 HPLC separation pump and a Waters 2414 refractive index detector. The mobile phase was tetrahydrofuran, and the flow rate was 0.3 mL/min. The peak areas of the test results were integrated, and fitted with the peak area integral curves of different molecular weight standards to obtain the M_{w}, Mₙ and PDI of the polymers. The molecular weight (Mₙ) and the polymerization degree superscripted with ^{b} were obtained by ¹H NMR test: the polymers were dissolved in deuterated chloroform (CDC₁₃) at a final concentration of 10 mg/mL, and the internal standard was tetramethylsilane (TMS). The data were obtained by scanning with a 400 MHz nuclear magnetic resonance spectrometer (Brooke, Germany). The number of hydrogen atoms corresponding to the PEG peak area integration was defined as 448, and the proton signal peaks of the hydrogen atoms at the remaining positions were integrates and calculated by comparing with the PEG peak area.

TABLE 3
Polymer materials synthesized in this
embodiment and their characterization

| | | | Molecular | | |
|---|---|---|---|---|---|
| | | | weight | | Polymeriza- |
| Polymer | M_{w} | Mₙ | distribution | Mn | tion Degree |
| materials | (g/mol)^{a} | (g/mol)^{a} | (PDI)^{a} | (g/mol)^{b} | (z)^{b} |
| P(C6-Me₁₀)₈₀ | 13500 | 8700 | 1.55 | 17900 | 69 |
| P(C6-Me₂₀)₈₀ | 12500 | 9800 | 1.27 | 17900 | 74 |
| P(C6-Me₃₀)₈₀ | 9800 | 7500 | 1.31 | 17200 | 78 |
| P(C6-E₁₀)₈₀ | 11400 | 8400 | 1.20 | 20200 | 80 |
| P(C6-E₂₀)₈₀ | 14800 | 11000 | 1.20 | 20400 | 87 |
| P(C6-E₃₀)₈₀ | 10000 | 7500 | 1.34 | 18100 | 78 |
| P(C6-Bu₁₀)₈₀ | 13600 | 10600 | 1.28 | 19600 | 75 |
| P(C6-Bu₂₀)₈₀ | 13200 | 10400 | 1.17 | 20400 | 79 |
| P(C6-Bu₃₀)₈₀ | 10100 | 12900 | 1.27 | 16200 | 71 |
| P(C6-Bn₅)₈₀ | 19300 | 13700 | 1.40 | 23000 | 86 |
| P(C6-Bn₁₀)₈₀ | 19600 | 14200 | 1.35 | 21800 | 82 |
| P(C6-Bn₂₀)₈₀ | 47600 | 36400 | 1.30 | 20300 | 80 |
| P(C6-Bn₃₀)₈₀ | 37000 | 27500 | 1.34 | 21500 | 84 |
| P(C6-Bn₄₀)₈₀ | 34200 | 26200 | 1.31 | 22000 | 88 |
| P(C6-H₅)₈₀ | 22900 | 16900 | 1.35 | 19800 | 85 |
| P(C6-H₁₀)₈₀ | 24400 | 15600 | 1.56 | 16700 | 73 |
| P(C6-H₂₀)₈₀ | 34100 | 44600 | 1.51 | 20600 | 90 |
| P(C6-IO₅)₈₀ | 55600 | 35900 | 1.54 | 22000 | 81 |
| P(C6-IO₁₀)₈₀ | 30300 | 19400 | 1.56 | 20100 | 73 |
| P(C6-IO₂₀)₈₀ | 35500 | 22100 | 1.64 | 19900 | 79 |
| P(C6-T₅)₈₀ | 40500 | 30800 | 1.31 | 21000 | 77 |
| P(C6-T₁₀)₈₀ | 32100 | 22300 | 1.43 | 19300 | 68 |
| P(C6-T₂₀)₈₀ | 44300 | 37100 | 1.24 | 21300 | 76 |
| P(C6-Ph₂₀)₈₀ | 34767 | 18657 | 1.86 | 23166 | 90 |
| P(C6-NPh₂₀)₈₀ | 20161 | 11653 | 1.67 | 22603 | 80 |

Note:
^{a}represents molecular weight (M_{w}, Mₙ) and molecular weight distribution were obtained by GPC test,
^{b}represents that Molecular weight (Mₙ) and polymerization degree were calculated by ¹H NMR.

### 2. Preparation of Polymer Nanomicelle Particles with R₁ being ethyl piperidine (C6) and R₂ being Different Hydrophobic Groups.

The polymer materials synthesized in this embodiment had hydrophilic polyethylene glycol and hydrophobic polymethacrylate, and could self-assemble into nano micelle particles in water. The specific operation is as follows: 50 mg of polymer material was added into a 5 mL sample bottle, and added 1 mL of N,N-dimethylformamide (DMF) to fully dissolve it for later use. 5 mL of sterile water was added into a 25 mL round bottom flask sterilized in advance. The flask was placed on a magnetic stirring table, and stirred at a speed of RPM=1500 r/min. The DMF solution of the polymer material was added dropwise into the round bottom flask using a pipette gun. Continued to stir at RPM=1000 r/min for 10 minutes, and then a dialysis bag with a molecular weight cut-off of 14000 was used to dialyze in 4 L of ultrapure water for 24 hours, changing the water every 1 hour fix the first 6 hours, and every 6 hours fix the next 18 hours. After dialysis, the nanoparticle solution was taken out with a pipette gun and quantified, and stored in a refrigerator at 4° C. 100 μL of the nanoparticle solution with a concentration of 5 mg/mL was diluted to 1 mL with the PBS solution (pH 7.4), and then was loaded into the potential/particle size special test cell of a nanoparticle size analyzer to test its particle size and potential. The test results are shown in Table 4. The polymer materials synthesized in this embodiment self-assembled in water to form nano micelles with particle size of about 20-80 nm, and the potential of about 0 mV, indicating electrical neutrality.

TABLE 4
pKa, particle size and potential characterization of
the series of polymer materials in solution at pH 7.4

| | | | Zeta potential |
|---|---|---|---|
| | | particle size | characterization |
| Polymer materials | pKₐ | (nm) | (mV) |
| P(C6-Me₁₀)₈₀ | 7.21 | 26.51 ± 2.99 | 0.30 ± 0.02 |
| P(C6-Me₂₀)₈₀ | 7.17 | 43.25 ± 0.68 | 0.56 ± 0.21 |
| P(C6-Me₃₀)₈₀ | 7.11 | 32.36 ± 0.82 | 0.65 ± 0.61 |
| P(C6-E₁₀)₈₀ | 7.14 | 23.86 ± 1.00 | 0.62 ± 0.16 |
| P(C6-E₂₀)₈₀ | 7.05 | 22.20 ± 0.34 | −0.15 ± 0.39 |
| P(C6-E₃₀)₈₀ | 7.01 | 27.88 ± 2.68 | 0.76 ± 0.20 |
| P(C6-Bu₁₀)₈₀ | 7.00 | 22.33 ± 4.57 | 0.36 ± 0.19 |
| P(C6-Bu₂₀)₈₀ | 6.83 | 23.53 ± 0.81 | −0.02 ± 0.18 |
| P(C6-Bu₃₀)₈₀ | 6.02 | 27.14 ± 0.40 | −0.24 ± 0.12 |
| P(C6-Bn₅)₈₀ | 7.14 | 28.41 ± 4.16 | 0.29 ± 0.13 |
| P(C6-Bn₁₀)₈₀ | 7.03 | 27.71 ± 2.49 | 0.24 ± 0.29 |
| P(C6-Bn₂₀)₈₀ | 6.86 | 55.82 ± 0.56 | −0.21 ± 0.18 |
| P(C6-Bn₃₀)₈₀ | 6.55 | 44.49 ± 1.64 | 1.26 ± 0.33 |
| P(C6-Bn₄₀)₈₀ | 6.28 | 73.96 ± 6.01 | −0.24 ± 0.17 |
| P(C6-H₅)₈₀ | 7.11 | 19.80 ± 3.80 | 1.75 ± 0.41 |
| P(C6-H₁₀)₈₀ | 6.91 | 40.90 ± 3.35 | 3.17 ± 0.20 |
| P(C6-H₂₀)₈₀ | 6.61 | 29.72 ± 1.98 | −0.29 ± 0.11 |
| P(C6-IO₅)₈₀ | 6.99 | 26.22 ± 2.22 | 3.05 ± 0.39 |
| P(C6-IO₁₀)₈₀ | 6.85 | 22.38 ± 1.46 | 1.34 ± 0.66 |
| P(C6-IO₂₀)₈₀ | 6.60 | 28.17 ± 0.90 | −0.01 ± 0.34 |
| P(C6-T₅)₈₀ | 7.06 | 25.69 ± 4.55 | 0.82 ± 0.37 |
| P(C6-T₁₀)₈₀ | 6.77 | 38.71 ± 1.57 | 3.30 ± 0.14 |
| P(C6-T₂₀)₈₀ | 6.59 | 27.19 ± 1.51 | −0.21 ± 0.43 |
| P(C6-Ph₂₀)₈₀ | 6.88 | 44.13 ± 2.99 | 1.20 ± 0.13 |
| P(C6-NPh₂₀)₈₀ | 6.81 | 58.07 ± 1.02 | 2.57 ± 1.49 |

### 3. Protonation of Polymer Materials with R₁ being ethylpiperidine(C6) and R₂ being Different Hydrophobic Groups at Different pH Values

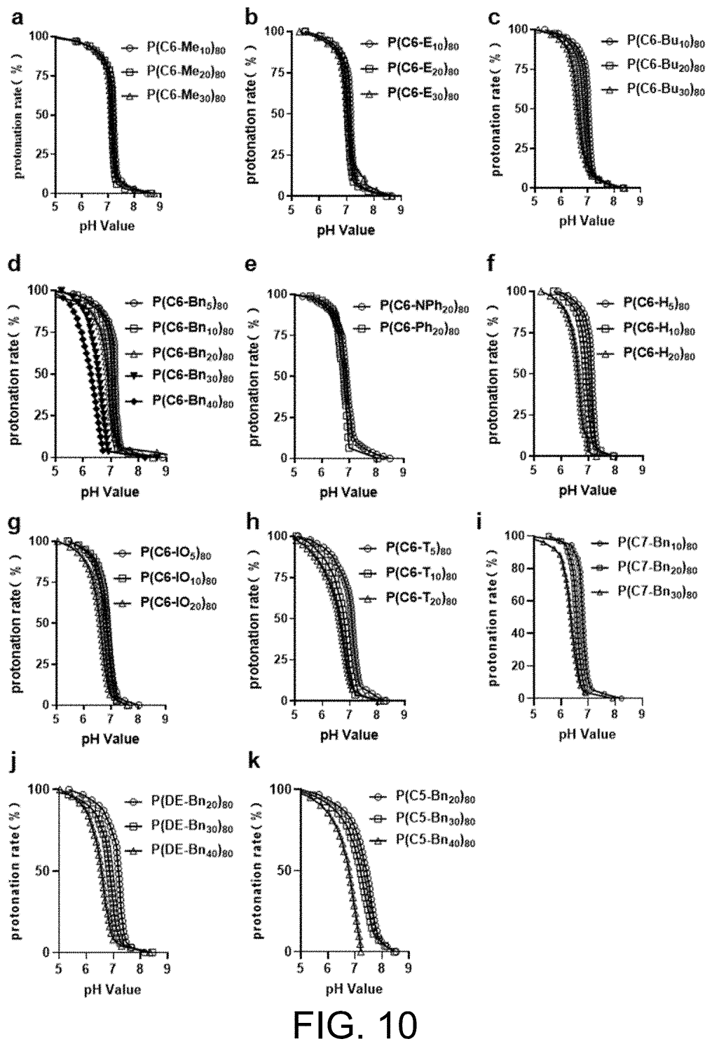

The polymer materials prepared in this embodiment were dissolved in an acidic solution containing 150 mM of NaCl to protonate all tertiary amines, and then the free hydrogen ions were neutralized with alkali. The protonation degree and pKa (ionization equilibrium constant) of the corresponding polymer materials were calculated by monitoring the pH change of the solution. The specific operations were as follows: 10 mg of polymer material was taken into a 20 mL sample bottle, HCl aqueous solution (10 mL, 0.01 M) was added to dissolve the polymer material into a clear solution. 1 M NaOH aqueous solution was added, 1˜10 L at a time, and a pH meter was used to monitor and record the pH change of the solution until the pH reached 11. The two extreme points of the first derivative of the pH titration curve were determined as fully protonated (protonation rate=1) and fully nonprotonated (protonation rate=0). When the protonation rate was 0.5, it proved that the molar amount of the protonated part and the unprotonated part of the polymer material in the solution system was the same. According to the definition of the dissociation equilibrium constant (pKa), the protonation rate of a polymer material equal to 0.5 is defined as the pKa of the polymer material. As shown in FIG. 10, the protonation rate of this type of polymer material changed rapidly with the change of and there was a great protonation rate jump in a very small pH range. It can be seen from the analysis that with the increase of the ratio of the hydrophobic structure of the polymer material, the pKa of the polymer material decreased in turn and had a linear relationship with the ratio of the methacrylate monomer containing hydrophobic groups. Therefore, the pH dependent protonation degree of a membranolytic polymer can be controlled by adjusting the ratio of the hydrophobic monomer to the tertiary amine in the polymer material structure.

### 4. Cytotoxicity of Polymeric Materials with R₁ being ethylpiperidine (C6) and R₂ being Different Hydrophobic Groups in Normal and Tumor Tissues at Characteristic pH

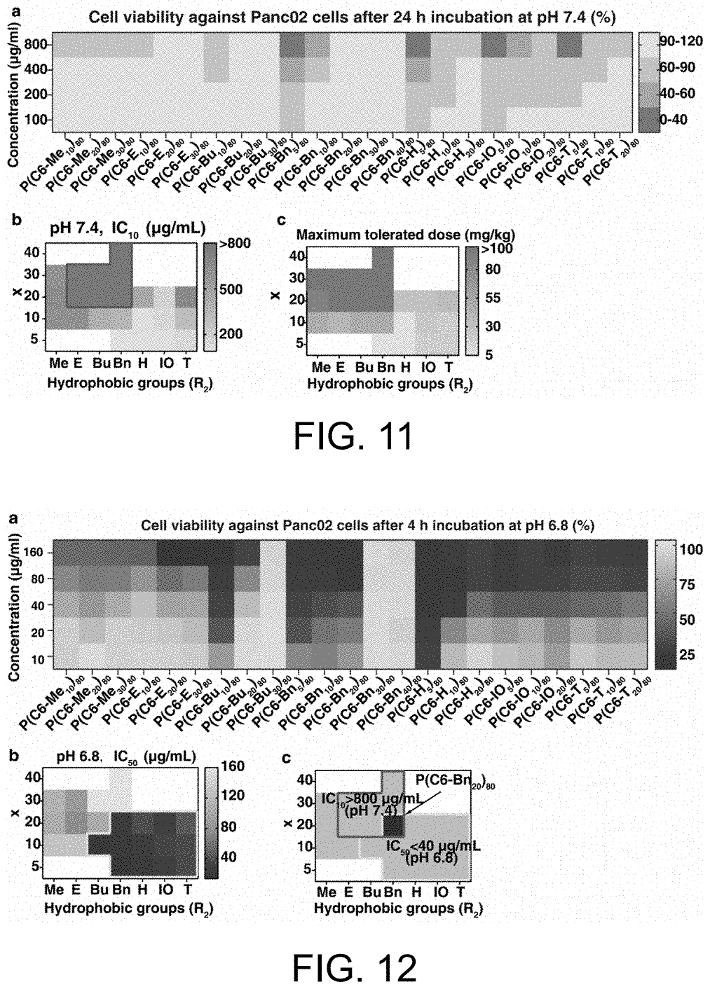

The cytotoxicity of the series of polymer materials prepared in this embodiment in normal tissues and tumor tissues at the characteristic pH was studied through cytotoxicology experiment, and the activated membranolytic polymers that showed low cytotoxicity in normal tissues but high cytotoxicity in tumor tissues were screened. MTT method (Microenzyme Reaction Colorimetry of Tetramethylazozolium) was applied to evaluate the killing effect of the polymer materials on tumor cells at pH 7.4 and pH 6.8. 6 mol/L HCl solution was used to adjust DMEM medium to pH=6.8 for standby. Panc02 cells (ATCC) were cultured in RPMI-1640 medium containing 10% (v/v) fetal bovine serum. At pH 7.4 and pH 6.8, the materials (nano micellar particles prepared in the second part of this embodiment) with different concentrations together with the cells (at a concentration of 1×10⁵/mL) were coincubated at 37° C. in a CO₂ incubator for 4 hours or 24 hours respectively. Then the original medium was discarded, and MTT (0.5 mg/mL, 100 μL) solution was added. The cells were placed in the CO₂ incubator to cultere at 37° C. for 4 hours, and then the culture medium was carefully aspirated away, and added 100 μL of DMSO to each well, and then the cells were placed on a shaker and shook at low speed for 10 minutes to fully dissolve the crystals. The absorbance value at OD 490 nm was measured by a microplate reader. The test results are shown in FIG. 11.

It can be seen from FIG. 11a that at pH 7.4, most polymer materials had weak effects on Panc02 cells. After incubating for 24 hours at high concentrations, the polymer materials had little toxicity to the cells, indicating that the polymer materials had no obvious cytotoxicity. According to the MTT results, the concentration of the material when the inhibition rate of Panc02 cells was 10% was calculated, which was defined as IC₁₀. It was analyzed graphically with different hydrophobic monomers and proportions of polymer materials, as shown in FIG. 11b. It can be found that the material which IC₁₀ to greater than 800 μg/mL, its proportion of hydrophobic monomer in all methacrylate monomers was not less than 20%, and the hydrophobic group R₂ was ethyl (E), butyl (Bu) or other shorter alkyl chain or benzyl (Bn) structure. When the hydrophobic group R₂ was a long alkyl chain such as hexyl (H), isooctyl (IO), tetradecyl (T), the cytotoxicity of the polymer was higher, and its IC₁₀ was lower, indicating that the increase of the hydrophobicity of the methacrylate monomer would increase the toxicity of the polymer.

All polymer materials were incubated with Panc02 cells at pH 6.8 for 4 hours to evaluate their killing ability under tumor acidity. The MTT results are as shown in FIG. 12a, wherein the darker the color, the stronger the killing ability of the polymer to cells. According to the MTT results, the concentration of the different polymer materials when the inhibition rate of Panc02 cells was 50% was calculated, which was defined as IC₅₀. It was analyzed graphically with different hydrophobic monomers and proportions of polymer materials, as shown in FIG. 12b. It can be found that the polymer materials with the hydrophobic group R₂ being a longer alkyl chain or benzyl group such as hexyl (H), isooctyl (IO), tetradecyl (T), etc, and with the proportion of hydrophobic monomers in all methacrylate monomers between 5% and 20%, had stronger cell killing ability.

Through the comprehensive analysis and mapping of IC₁₀ at pH 7.4 and IC₅₀ at pH the optimal type and proportion of hydrophobic monomer were obtained.

As shown in FIG. 11b, the block represents the polymer types with IC₁₀ greater than 800 μg/mL; and as shown in FIG. 12b, the block represents the polymer types with IC₅₀ less than 40 μg/mL. Overlapping the two blocks (as shown in FIG. 12c), the intersection was polymer P(C6-Bn₂₀), which had low cytotoxicity (IC₁₀>800 μg/mL) under normal physiological conditions (pH=7.4)), and had strong cell killing ability (IC₅₀=23.23 μg/mL) in the tumor acid environment (pH=6.8).

The toxicity of these polymer materials was further studied by testing the maximum lethal dose (MTD) in mice. 5-week-old female ICR mice (Hunan Shrek Jingda Experimental Animal Co., Ltd.) were divided into 6 groups with similar average weight, with 6 mice in each group. The corresponding materials were P(C6-Me_{y})₈₀, P(C6-Bu_{y})₈₀, P(C6-E_{y})₈₀, P(C6-Bn_{y})₈₀, P(C6-H_{y})₈₀, and P(C6-T_{y})_{80,} wherein, y refers to the percentage of methacrylate monomers containing hydrophobic groups in all methacrylate monomers. Each material was administered starting from 10 μg/g body weight, and the same dose was administered to three female ICR mice; If no experimental animal died after 24 hours, the dosage was increased by 5 μg/g and administered again until the death of the mice occurred. The maximum dose that did not cause death in mice was recorded as the maximum tolerable dose of ICR mice, and potte. As shown in FIG. 11c, when the hydrophobic group R₂ was a shorter alkyl chain such as ethyl (E) and butyl (Bu), or benzyl (Bn), and the proportion of the hydrophobic monomers in all methacrylate monomers was not less than 20%, the MTD of these polymers was greater than 100 mg/kg.

The relevant data are shown in Table 5.

TABLE 5
The concentrations of series of polymer materials killing
10% cells (IC₁₀) after incubating with Panc02 cells at pH
7.4 for 24 hours, the concentrations of series of polymer materials
killing 50% cells (IC₅₀) after incubating with Panc02 cells
at pH 6.8 for 4 hours, and the maximum lethal dose (MTD)
after tail vein injection into ICR mice

| Polymer | IC₁₀ | IC₅₀ | MTD |
|---|---|---|---|
| materials | (μg/mL) | (μg/mL) | (mg/kg) |
| P(C6-Me₁₀)₈₀ | 614.93 | 117.40 | 50 |
| P(C6-Me₂₀)₈₀ | 643.55 | 80.00 | 90 |
| P(C6-Me₃₀)₈₀ | 607.30 | 88.10 | >100 |
| P(C6-E₁₀)₈₀ | 611.86 | 100.00 | 40 |
| P(C6-E₂₀)₈₀ | >800.00 | 80.00 | >100 |
| P(C6-E₃₀)₈₀ | >800.00 | 80.00 | >100 |
| P(C6-Bu₁₀)₈₀ | 400.00 | 80.00 | 50 |
| P(C6-Bu₂₀)₈₀ | >800.00 | 51.83 | >100 |
| P(C6-Bu₃₀)₈₀ | >800.00 | 30.00 | >100 |
| P(C6-Bn₅)₈₀ | 90.00 | 70.43 | 5 |
| P(C6-Bn₁₀)₈₀ | 358.48 | 18.94 | 50 |
| P(C6-Bn₂₀)₈₀ | >800.00 | 17.68 | >100 |
| P(C6-Bn₃₀)₈₀ | >800.00 | >160.00 | >100 |
| P(C6-Bn₄₀)₈₀ | >800.00 | >160.00 | >100 |
| P(C6-H₅)₈₀ | 100.00 | 11.58 | 5 |
| P(C6-H₁₀)₈₀ | 100.00 | 24.70 | 5 |
| P(C6-H₂₀)₈₀ | 455.90 | 37.52 | 30 |
| P(C6-IO₅)₈₀ | 100.00 | 31.91 | 20 |
| P(C6-IO₁₀)₈₀ | 144.80 | 33.30 | 25 |
| P(C6-IO₂₀)₈₀ | 170.08 | 25.08 | 30 |
| P(C6-T₅)₈₀ | 142.26 | 36.14 | 20 |
| P(C6-T₁₀)₈₀ | 307.29 | 36.06 | 20 |
| P(C6-T₂₀)₈₀ | 672.28 | 33.60 | 35 |

It can be found from this embodiment that when the hydrophobic group was benzyl and and the proportion of its hydrophobic monomers in all methacrylate monomers was 20% , the polymer material P (C6-Bn₂₀)₈₀ had a low cytotoxicity (IC₁₀>800 μg/mL) under normal physiological conditions (pH=7.4), and in the slightly acidic environment of tumor (pH=6.8), it had a strong cell killing ability and in vivo toxicity (IC₅₀=23.23 μg/mL, MTD>100 mg/kg), indicating that P (C6-Bn₂₀)₈₀ had the strongest selectivity at pH 7.4 and pH 6.8.

### Embodiment 2

### 1, Preparation and Characterization of Polymers with R₂ being benzyl and R₁ being Different Tertiary Amines

In this embodiment, a series of polymer materials as shown in Formula (III) were synthesized by RAFT polymerization, and the reaction equation is as follows:

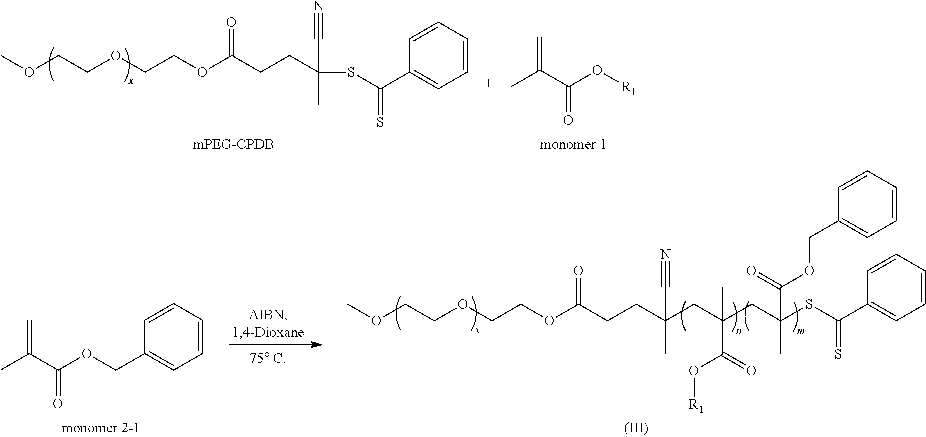

wherein R₁ is

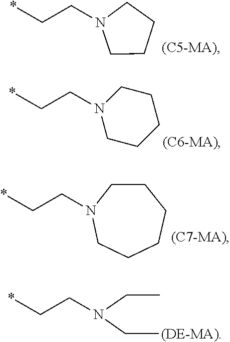

The specific steps are as follows:

200 mg of mPEG113-CPDB was dissolved in 1,4-dioxane and placed in a reaction vessel, with a total molar ratio of mPEG-CPDB to monomer being 1:80. Monomer 1 and benzyl methacrylate (Bn-MA) were added according to different molar ratios of R₁ and R₂ (80:20, 75:20, 70:30, 63:35, 60:40). After mixed evenly, added 2 mg of initiator AIBN and froze into solid and then reduced the pressure, and the temperature was restored to room temperature to release the oxygen from the reaction solution and froze again. Repeated this several times until there was no oxygen in the reaction vessel, and then filled with high-purity nitrogen and seal the reaction for 18-24 hours. After the reaction was completed, the reaction was terminated by freezing in liquid nitrogen. The reaction solution was melted and dropped into n-hexane to obtain an orange-yellow polymer material.

The abbreviation of the polymer materials prepared in this embodiment and the feeding ratios are shown in Table 6, wherein the reactant R₁ refers to methacrylate monomer 1 with side group containing tertiary amine, and the reactant R₂ refers to methacrylate monomer 2 containing hydrophobic groups (Bn-MA in this embodiment).

TABLE 6
Synthesis conditions and pKa of series of polymer materials

| | | Reactant | | | |
|---|---|---|---|---|---|
| Abbreviations | Chain | R₁ | R₂ | Feeding ratio | |
| of polymer | transfer agent | (tertiary | (hydrophobic | (Chain transfer | |
| materials | (PEG-CPDB) | amine) | groups) | agent:R₁:R₂) | pKa |
| P(C5-Bn₂₀)₈₀ | mPEG₁₁₃ | C5-MA | BnMA | 1:64:16 | 7.40 |
| P(C5-Bn₃₀)₈₀ | mPEG₁₁₃ | C5-MA | BnMA | 1:56:24 | 7.20 |
| P(C5-Bn₃₅)₈₀ | mPEG₁₁₃ | C5-MA | BnMA | 1:52:28 | 7.05 |
| P(C5-Bn₄₀)₈₀ | mPEG₁₁₃ | C5-MA | BnMA | 1:48:32 | 6.77 |
| P(C7-Bn₁₀)₈₀ | mPEG₁₁₃ | C7-MA | BnMA | 1:72:8 | 6.79 |
| P(C7-Bn₂₀)₈₀ | mPEG₁₁₃ | C7-MA | BnMA | 1:64:16 | 6.59 |
| P(C7-Bn₃₀)₈₀ | mPEG₁₁₃ | C7-MA | BnMA | 1:56:24 | 6.36 |
| P(DE-Bn₂₀)₈₀ | mPEG₁₁₃ | DE-MA | BnMA | 1:64:16 | 7.40 |
| P(DE-Bn₂₅)₈₀ | mPEG₁₁₃ | DE-MA | BnMA | 1:60:20 | 7.11 |
| P(DE-Bn₃₀)₈₀ | mPEG₁₁₃ | DE-MA | BnMA | 1:56:24 | 7.00 |
| P(DE-Bn₄₀)₈₀ | mPEG₁₁₃ | DE-MA | BnMA | 1:48:32 | 6.55 |

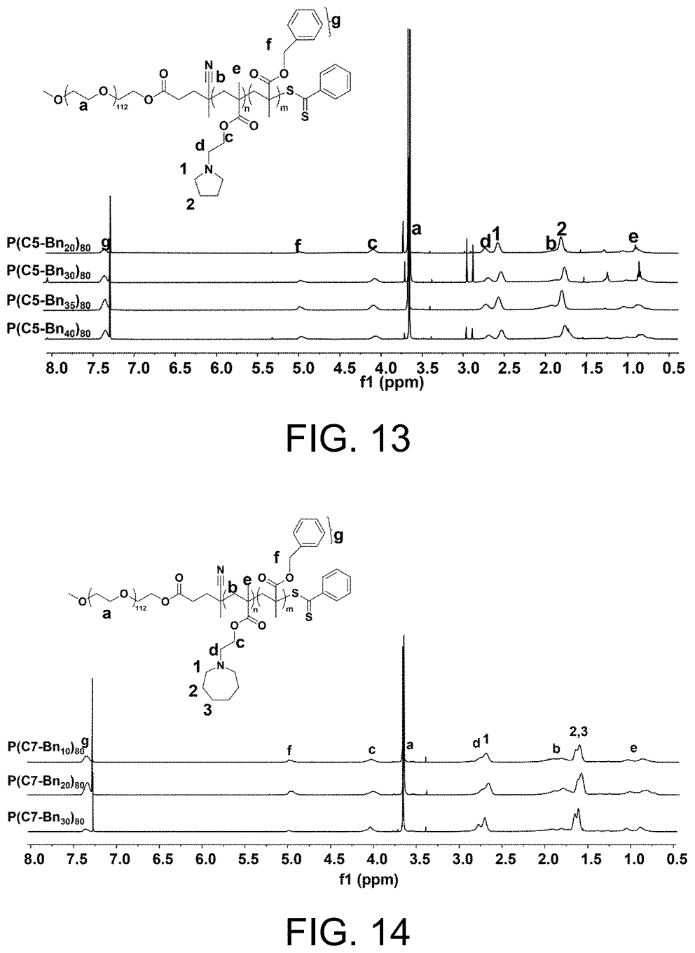

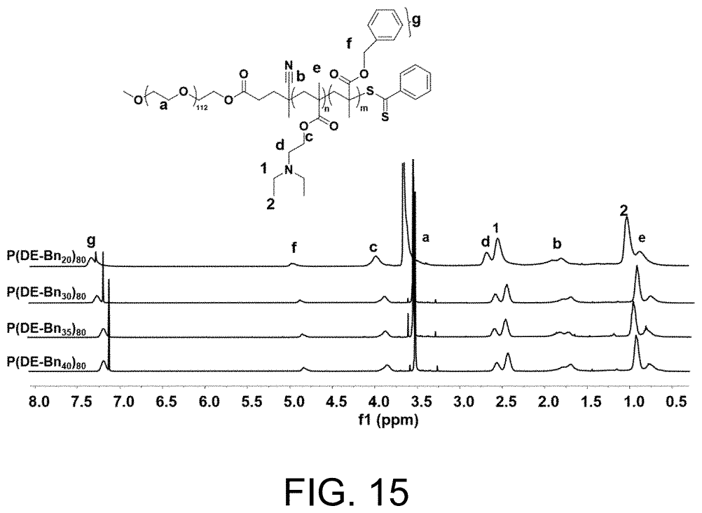

The NMR spectra of the polymer materials prepared in this embodiment are shown in FIG. 13-FIG. 15, wherein 3.66 ppm (a) is the proton signal peak of the hydrogen atom (—CH₂CH₂O—) on the polyethylene glycol chain, and 1.85 ppm (b) is the proton signal peak of the hydrogen atom (—CH₂—) on the polymer main chain; 0.9˜1.05 ppm (e) is the proton signal peak of methyl hydrogen atom (—CH₃) on the side chain, 2.62 ppm (d, —CH₂N—), 4.10 ppm (c, —OCH₂—) are the proton signal peaks of hydrogen atom (—CH₃) on the side chain, 5.00 ppm (f, —OCH₂—), 7.34 ppm [g, (—CH═)₅] are the proton signal peaks of hydrogen atom on the benzyl group of the hydrophobic side chain. For P (C5 Bnₓ), the characteristic peaks of 2.55 ppm (1, —NCH₂—) and 1.78 ppm (2, —CH₂—) are the proton signal peaks of the hydrogen atom on C5 heterocycle. For P (C7-Bnₓ), the characteristic peaks of 2.65 ppm (1, —NCH₂—), 1.68 ppm (2, —CH₂—) and 1.60 (3, —CH₂—) are the proton signal peaks of the hydrogen atom on the C7 heterocycle, For P (DE-Bnₓ), the characteristic peaks of 2.50 ppm (1, —NCH₂—) and 1.00 ppm (2, —CH₃) are the proton signal peaks of the hydrogen atoms on DE alkyl chain. The NMR data showed that the series of polymer materials with controllable molecular weight and molar ratio of R₁ to R₂, which were shown in the above-mentioned Formula (III), were successfully synthesized.

### 2. Preparation of Polymer Nanoparticles with R₂ being benzyl and R₁ being Different Tertiary Amines

The specific operations for the self-assembly of the polymer materials synthesized in this embodiment to form nanoparticles in water are as follows: 50 mg of the polymer material was added into a 5 mL sample bottle, added 1 mL of N,N-dimethylformamide (DMF) to fully dissolve it for later use. 5 mL of sterile water was added into a 25 mL round-bottomed flask sterilized in advance. The flask was placed on a magnetic stirring table, and stirred at a speed of RPM=1500 r/min. The DMF solution of the polymer material was added dropwise into the round-bottomed flask using a pipette gun. Continued to stir at RPM=1000 r/min for 10 min, and then a dialysis bag with molecular weight cut-off of 14000 was used to dialyze in 4 L of ultrapure water for 24 hours, changing the water every 1 hour for the first 6 hours, and every 6 hours for the next 18 hours. After dialysis, the nanoparticle solution was taken out with a pipette gun and quantified, and stored in a refrigerator at 4° C. Then particle size potentiometer was used to test the particle size and potential of the products. The test results showed that the polymer materials synthesized in this embodiment self-assembled in water to form nano micelles with a particle size of about 50 nm-60 nm, and the potential was about 0 mV indicating electrical neutrality.

### 3. Similarly, as shown in FIG. 10i-FIG. 10k, through Titration Experiments, it was Proved that the Degree of Protonation of this Kind of Polymers also Jumped with pH, and Their pKa Decreased with the Increase of the Hydrophobicity of Tertiary Amine and the Proportion of Benzyl Impurities.

### 4. Cytotoxicity of Polymeric Materials with R₂ being Benzyl and R₁ being Different Tertiary Amines at Characteristic pH of Normal and Tumor Tissues

The experimental method was the same as that in Embodiment 1.

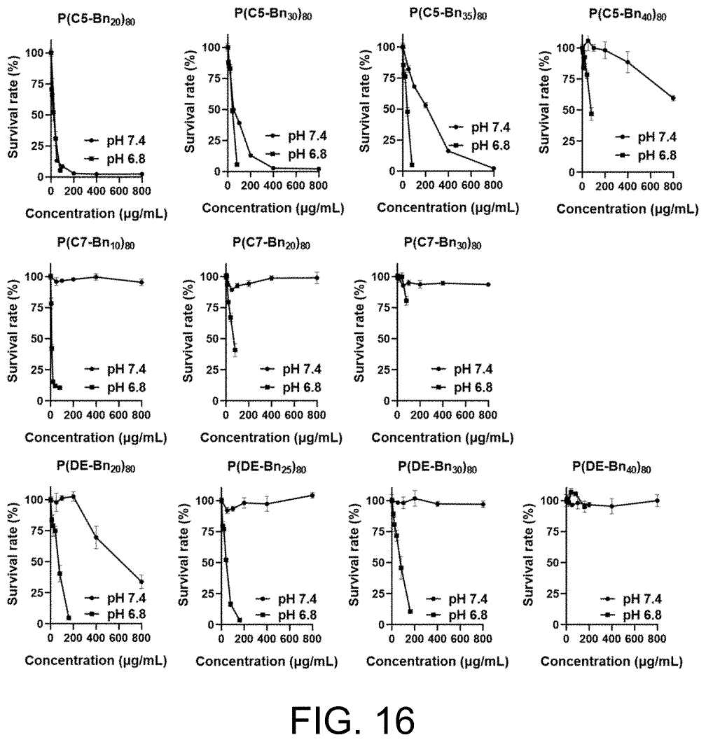

The experimental results are shown in FIG. 16 and Table 7: Nanoparticles of the polymer materials with a certain proportion of hydrophobic side chains containing benzyl groups selectively killed tumor cells at different pH values, and showed stronger cytotoxicity at pH 6.8. The polymer materials P(C7-Bn₂₀)₈₀, P(C7-Bn₃₀)₈₀, P(C7-Bn₄₀)₈₀ and P(DE-Bn₂₅)₈₀, P(DE-Bn₃₀)₈₀, P(DE-Bn₄₀)₈₀ showed lower cytotoxicity at pH 7.4, with IC₁₀>800 μg/mL. Further studies on the MTD of these polymer nanoparticles on ICR mice showed that when the tertiary amine was DE, although the polymer nanoparticles showed lower cytotoxicity at pH 7.4, they still had certain toxicity in vivo. Based on the above results, the polymer material P (C6-Bn₂₀)₈₀ nanoparticles had the strongest selectivity at pH 7.4 and pH 6.8, and with higher MID in mice.

TABLE 7
The concentrations of series of polymer materials killing
10% cells (IC₁₀) after incubating with Panc02 cells at pH
7.4 for 24 hours, the concentrations of series of polymer materials
killing 50% cells (IC₅₀) after incubating with Panc02 cells
at pH 6.8 for 4 hours, and the maximum lethal dose (MTD)
after tail vein injection into ICR mice

| Abbreviation | | | |
|---|---|---|---|
| of polymer | IC₁₀ | IC₅₀ | MTD |
| materials | (pH 7.4, μg/mL) | (pH 6.8, μg/mL) | (mg/kg) |
| P(C6-Bn₂₀)₈₀ | >800.00 | 23.23 | >100 |
| P(C5-Bn₂₀)₈₀ | 5.13 | 43.55 | N.D. |
| P(C5-Bn₃₀)₈₀ | 12.78 | 76.33 | N.D. |
| P(C5-Bn₃₅)₈₀ | 24.95 | 77.56 | N.D. |
| P(C5-Bn₄₀)₈₀ | 336.67 | 151.34 | N.D. |
| P(C7-Bn₂₀)₈₀ | >800.00 | 67.00 | >100 |
| P(C7-Bn₃₀)₈₀ | >800.00 | >160.00 | >100 |
| P(C7-Bn₄₀)₈₀ | >800.00 | >160.00 | >100 |
| P(DE-Bn₂₀)₈₀ | 255.00 | 69.22 | 10 |
| P(DE-Bn₂₅)₈₀ | >800.00 | 56.56 | 40 |
| P(DE-Bn₃₀)₈₀ | >800.00 | 73.22 | 50 |
| P(DE-Bn₄₀)₈₀ | >800.00 | >160.00 | 80 |

### Embodiment 3 Effect of Molecular Weight on the Activity of Polymer Materials

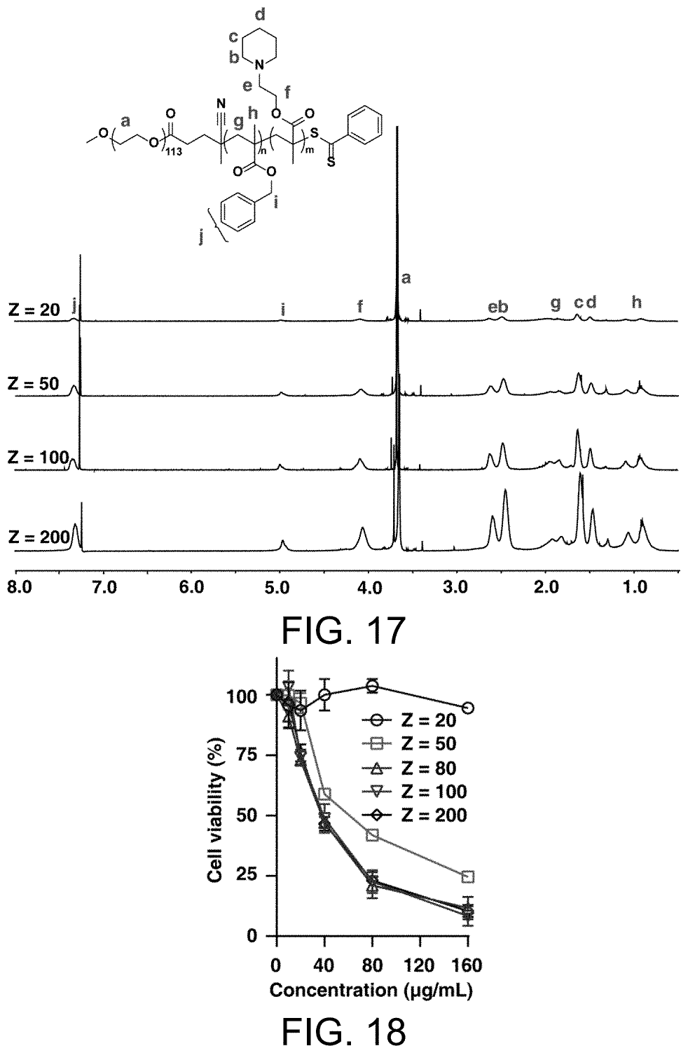

1. A series of P (C6-Bn₂₀) with different polymerization degrees (Z, which is the sum of polymerization degrees containing C6 and Bn segments) were synthesized and characterized in this embodiment using the method of embodiment 1. The specific NMR characterization is shown in FIG. 17. According to the proton signal peak of hydrogen atom on the polyethylene glycol chain segment at 3.66 ppm (a, —CH₂CH₂O—), and the proton signal peaks of hydrogen atoms on the side chains at 2.48 ppm (b, —NCH₂—), 1.62 ppm (c, —CH₂—), 1.47 ppm (d, —CH₂—), the integral area and degree of polymerization were calculated.

MTT method (the specific method was the same as that in embodiment 1) was used to test the kilning ability of P (C6-Bn₂₀) nanoparticles with different polymerization degrees (the preparation method was the same as that in embodiment 1) to Panc02 cells at pH 6.8. The test results as shown in FIG. 18 showing that: when the polymerization degree was 20, the killing ability of P(C6-Bn₂₀)₂₀ to Panc02 cells was weak; with the increase of polymerization degree, the killing ability was gradually enhanced. When the polymerization degree was 80 or above, the molecular weight had no effect on the killing effect of cells.

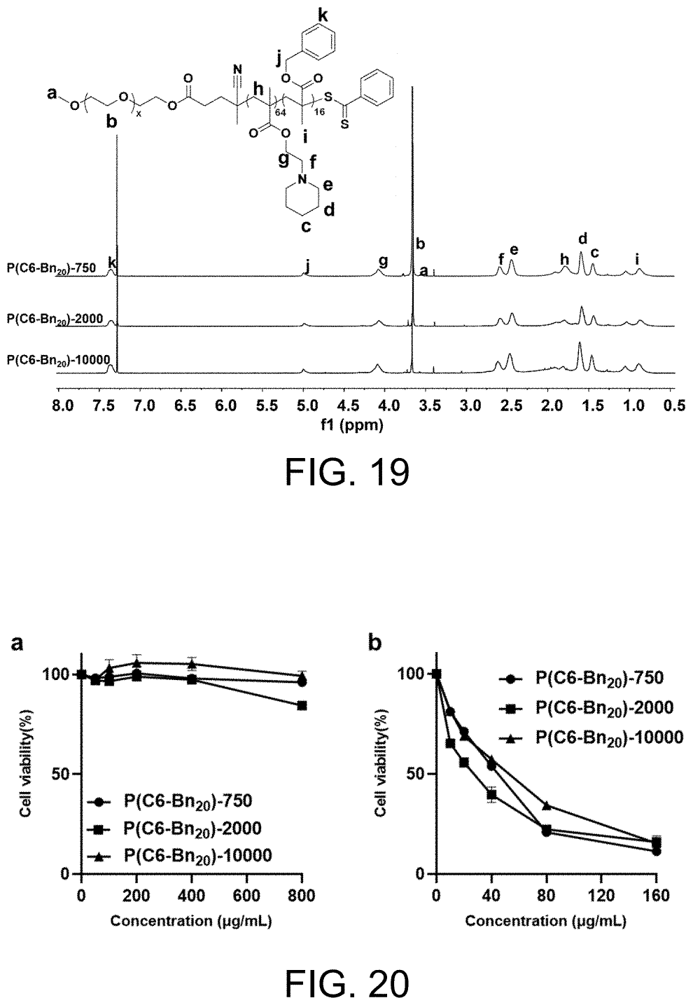

2. In this embodiment, a series of mPEGs with different molecular weights (molecular weights of 750, 2000 and 10000 respectively, corresponding to the values of x in the structural formula of 16, 44 and 224 respectively) were used as chain transfer agents to synthesize the polymer materials shown in Formula I with reference to the method of embodiment 1. According to the molecular weights of mPEGs, they were named as P (C6-Bn₂₀)-750, P (C6-Bn₂₀)-2000, and P (C6-Bn₂₀)-10000 respectively. The NMR characterization of this series of polymer materials is shown in FIG. 19, which proved that the designed polymer was successfully obtained. The cytotoxicity of the series of polymer material nanoparticles (the preparation method is the same as that in embodiment 1) was tested at pH 7.4 and pH 6.8 (the specific method is the same as that in embodiment 1). The results showed that different PEG lengths had little effect on its cytotoxicity at pH 7.4. After 24 hours of incubation at the concentration of 800 μg/mL, its cytotoxicity was lower (as shown in FIG. 20a), and the effect on its cytotoxicity at pH 6.8 was not significant (as shown in FIG. 20b).

### Embodiment 4: Physicochemical Properties of P (C6-Bn₂₀) Polymer Nanoparticles

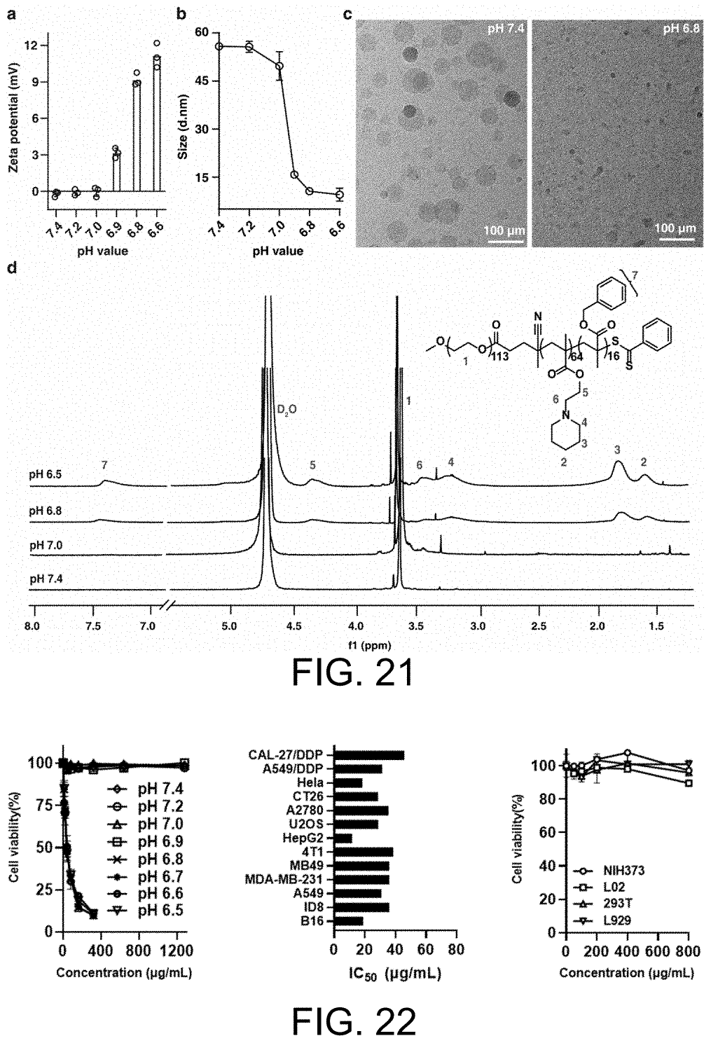

50 mg of the polymer material prepared in embodiment 1 was added into a 5 mL sample bottle, and added 1 mL of N,N-dimethylformamide (DMF) to fully dissolve it for later use. 5 mL of sterile water was added into a 25 mL round-bottomed flask sterilized in advance. The flask was placed on a magnetic stirring table, and stirred at a speed of RPM=1500 r/min. The DMF solution of the polymer material was added dropwise into the round-bottomed flask using a pipette gun. Continued to stir at RPM=1000 r/min for 10 min, and then a dialysis bag with a molecular weight cut-off of 14000 was used to dialyze in 4 L of ultrapure water for 24 hours, changing the water every 1 hour for the first 6 hours, and every 6 hours for the next 18 hours. After dialysis, the nanoparticle solution was taken out with a pipette gun and quantified, and stored in a refrigerator at 4° C. 100 μL of the nanoparticle solution with a concentration of 5 mg/mL was diluted to 1 mL with the PBS solution of corresponding pH value, and then the solution was loaded into the special potential/particle: size special test cell of a nanoparticle size analyzer, and nano meter and Zeta potentiometer were used to test the particle size and potential. As shown in FIG. 21a-FIG. 21b, when pH=7.4, the particle size was 55.8 nm, and the potential was only −0.2 mV; when the pH was 6.9, the particle size and potential of the nanoparticles began to change, the potential was 3.15 mV, and the particle size was 15.78 nm, indicating that the structure of the nanoparticles changed dramatically due to the protonation of the tertiary, amine structure. Furthermore, when pH<6.8, more tertiary amine structures in the nanoparticles were transformed into positive electricity and forming a cationic region. After the structural transformation, the particle size was about 10 nm, and the potential was greater than 9 mV. The morphologies of nanoparticles under two pH conditions were observed through TEM. As shown in FIG. 21c, P (C6-Bn₂₀) had a complete particle morphology under pH 7.4, while when pH was 6.8, the nanoparticles were basically transformed into small particles with a particle size of about 10 nm.

The combination of tertiary amine units in the polymers with free hydrogen ions in solution will lead to the conversion of polymers from hydrophobic to hydrophilic, thereby exposing part of the hydrophobic cores. The protonation process of the polymers can be confirmed by measuring the signal intensity of the hydrogen in hydrophobic core by NMR. The specific operations were as follows: 1.8 mg of the polymer solid and 0.054 g of NaCl were added into 6 mL of heavy water, and vortexed to disperse the solid as much as possible. Then 7 μL of deuterated hydrochloric acid heavy water solution was added, and ultrasonic treated until the polymer was completely dissolved to obtain a dear solution. 0.1 mol/L solution was prepared with heavy water and sodium deuterium oxide, added 1 μL of deuterium oxidizes sodium heavy aqueous solution to the high molecular weight aqueous solution successively, and the pH value was measured by a pH meter. When the pH was 6.0, 6.5, 6.8, 7.0, 7.4, respectively took 600 μL of sample into the nuclear magnetic tube to determine the hydrogen spectrum using a nuclear magnetic resonance. P (C6-Bn₂₀) so at three different pH values was characterized in the solution system by NMR. It can be seen from FIG. 21d that P (C6-Bn₂₀)₈₀ has no signal of hydrogen atom on the tertiary amine side chain and no signal of hydrogen atom on the benzene ring at pH 7.4 and pH 7.0, However, when the pH dropped to 6.8 and below, the characteristic signal peaks of the hydrogen atom on the tertiary amine side chain appeared on the hydrogen spectrum: 1.59 ppm (2), 1.80 ppm (3), 3.18 ppm (4), and the characteristic peak of the hydrogen atom on the benzene ring: 7.42 ppm (7). These results proved that the tertiary amine and benzene ring structures were sealed in the hydrophobic core at pH 7.4. While under the acidic condition of tumor, the strong charge repulsion between molecules and the decrease of hydrophobicity made the aggregates loose and exposed the chain segments capable of interacting with the cell membrane.

MTT method (the specific method was the same as in embodiment 1) was used to detect the concentration dependent cytotoxicity of P (C6-Bn₂₀) so to panc02 cells at different pH values. The results as shown in FIG. 22a: showing that the toxicity of P (C6-Bn₂₀)₈₀ was negligible in the pH range of 7.4-6.9, and in the pH range of 6.8-6.5, when the concentration was 40 μg/mL, the cell survival rate was lower than 50% after 4 hours of incubation.

At the same time, the cytotoxicity of P (C6-Bn₂₀)₈₀ to a variety of cancer cells at different pH values was detected by MTT method (the specific method was the same as in embodiment 1). At pH 6.8, P (C6-Bn₂₀)₈₀ had a good killing effect on a variety of cancer cells, including drug resistant cells (A549/DDP and CAL-27/DDP). The IC₅₀ values of this material against various tumor cells were all lower than 50 μg/mL at pH 6.8 (as shown in FIG. 22b). At the same time, it had no obvious toxicity to normal cells incubated at high concentration for 24 hours at pH 7.4 (as shown in FIG. 22c).

### Embodiment 5: Study on the Verification of the Mechanism of P (C6-Bn₂₀)₈₀ Polymer Material Killing Cells

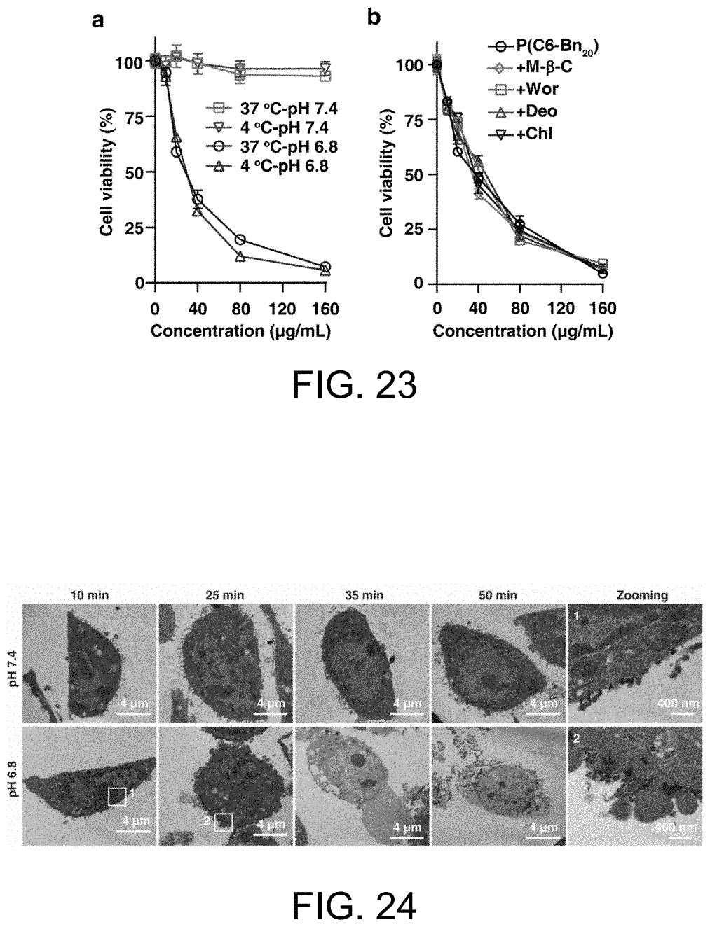

(1) This embodiment further studied whether the endocytosis of P C6-Bn₂₀)₈₀ polymer material affected its killing effect on Panc02 cells. Cells were treated with endocytosis inhibitors or co-incubated with the polymer material at 4° C., As shown in FIG. 23a, under the conditions of pH 7.4 and 6.8, Panc02 cells and P (C6-Bn₂₀)₈₀ nanoparticles were placed at 4° C. for 30 minutes in advance, and then were co-incubated again for 4 hours to detect the cell survival rate. Cells and nanoparticles were co-incubated at 37° C. as controls. The results showed that the change of temperature did not affect the killing effect of the polymer nanoparticles on cells. Since low temperature can inhibit endocytosis, it can be speculated that P (C6-Bn₂₀)₈₀ does not act through the classical endocytosis pathway. In addition, as shown in FIG. 23b, after the cells were pretreated with different endocytosis inhibitors and co-incubated with P (C6-Bn₂₀)₈₀, the killing effect of the polymer were not affected, which was consistent with that of the control group. It was further proved that P (C6-Bn₂₀)₈₀ could kill cells only by destroying the membrane structure of cells without being affected by endocytosis.

(2) 5×10⁶ suspension Panc02 cells were added to a 100 mm cell culture dish and incubated in a 5% CO₂ incubator at 37° C. for 24 hours to adhere to the wall, and then the medium was replaced with DMEM medium containing 100 μg/mL P (C6-Bn₂₀)—Fe with pH 6.8 or 7.4. Cells were collected with a cell scraper at different time points, fixed with 2.5% glutaraldehyde for 4 hours, and then rinsed with PBS three times, 10-15 minutes each time. Then osmium tetroxide was used to fix cells for 1-2 hours, and the cells were rinsed for three times with PBS, and each time for 10-15 minutes. Ethanol with gradient concentrations (25%, 50%, 75%, 90%, 100%) were used to dehydrate the cells in a gradient manner, by soaking the cells with corresponding concentration ethanol for 10-15 minutes in turn and then soaking them in anhydrous acetone for 10-15 minutes. Then the cells were immersed in acetone: embedding solution=3:1 (v/v) solution for 0.5 hours, and in acetone: embedding solution=1:1 (v/v) solution for 4 hours, and stored in pure embedding solution at 4° C. overnight. Then the samples were put into the embedding plate and baked at 37° C. for 24 hours, and then at 60° C. for 48 hours. The samples were cut into thin slices about 100 nm thick with an ultramicrotome (EM UC7, Leica), stained with uranium dioxane acetate for 20 minutes, stained with lead citrate for 12 minutes, and then observed with a transmission electron microscope. As shown in FIG. 24, after Panc02 cells were incubated with P(C6-Bn₂₀)₈₀ at pH 7.4 for 50 minutes, there were no changes in the cell membrane structure being observed, and the cell structures remained complete. In contrast, when the pH was 6.8, the nanoparticles attachment to the cell membrane was observed after only 10 minutes of co-incubation. When the incubation time was extended to 25 minutes, it was found that the cells had obvious morphological changes, and small bubbles appeared on the membrane. It is further demonstrated by magnification that the nanoparticles caused bubbling of the cell membrane. After the co-incubation time exceeded 35 minutes, the cellular content flowed out.

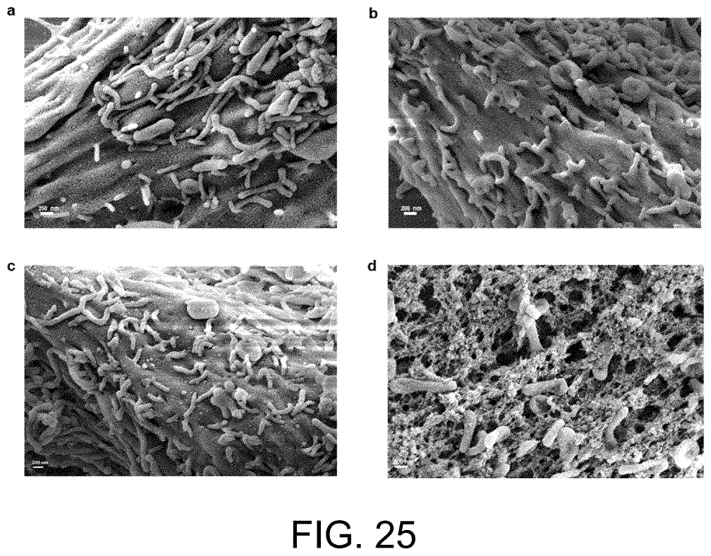

(3) Panc02 cells were cultured for 24 hours on a patch. After the cells were fixed on the patch, the patch was divided into 6 groups, namely, the pH 7.4 and pH 6.8 control groups treated with PBS, and the experimental group added with 50 μg/mL of P (C6-Bn₂₀). After 2 hours of treatment, cells were fixed with 2.5% glutaraldehyde solution for 20 minutes. Then washed three times with cold PBS, dehydrated by ethanol with mass concentrations of 25%, 50%, 75%, 90%, and 100% in a gradient manner and with each concentration gradient 20 minutes. After dehydration, the critical point drying method was used for drying. The vacuum spraying instrument was used to spray carbon and gold evenly, and the field emission scanning electron microscope (SEM) was used for observation (manufacturer: Carl Zeiss A G, model: Merlin). As shown in FIG. 25, P (C6-Bn₂₀)₈₀ material showed strong membranolytic activity in slightly acidic environment. FIG. 25a and b showed the electron microscopic photos of mouse pancreatic cancer cells (Pane-02 cells) at pH 7.4 and pH 6,8, respectively. FIG. 25c showed that the cell membrane was not damaged after treated with P (C6-Bn₂₀)₈₀ at pH 7.4, and d showed that the cell membrane was completely broken after treated with P (C6-Bn₂₀)₈₀ at pH 6.8.

### Embodiment 6: Tumor Inhibition Experiment of P (C6-Bn20)80 Polymer Material In Vivo

The tumor inhibition effect of P (C6-Bn₂₀)₈₀ was verified by the subcutaneous model of C57 mice with pancreatic cancer (Panc-02 cells), C57 mice with melanoma (B16-F10 cells), BALB/c mice with colorectal cancer (CT-26 cells), and BALB/c Nude mice with human lung cancer (A549 cells). The operation is as follows:

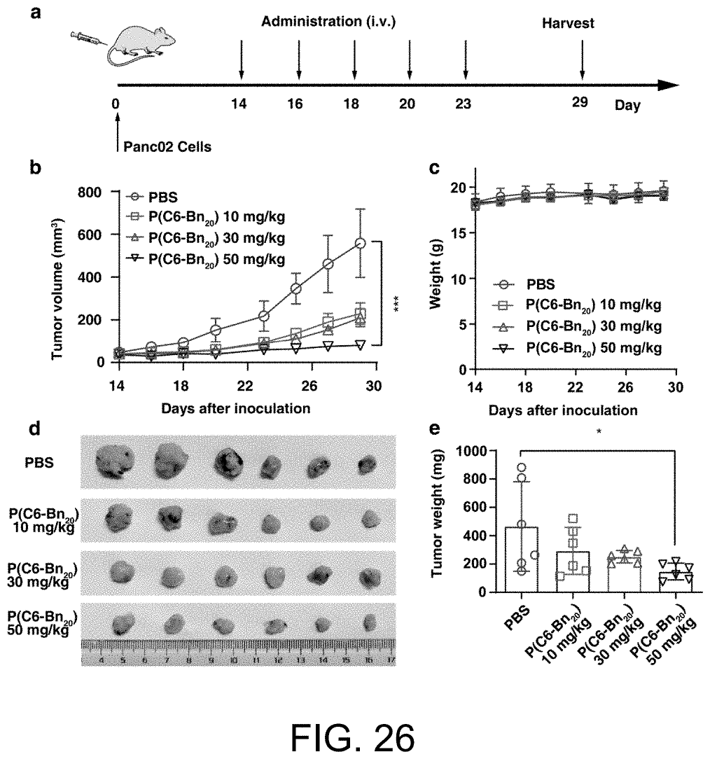

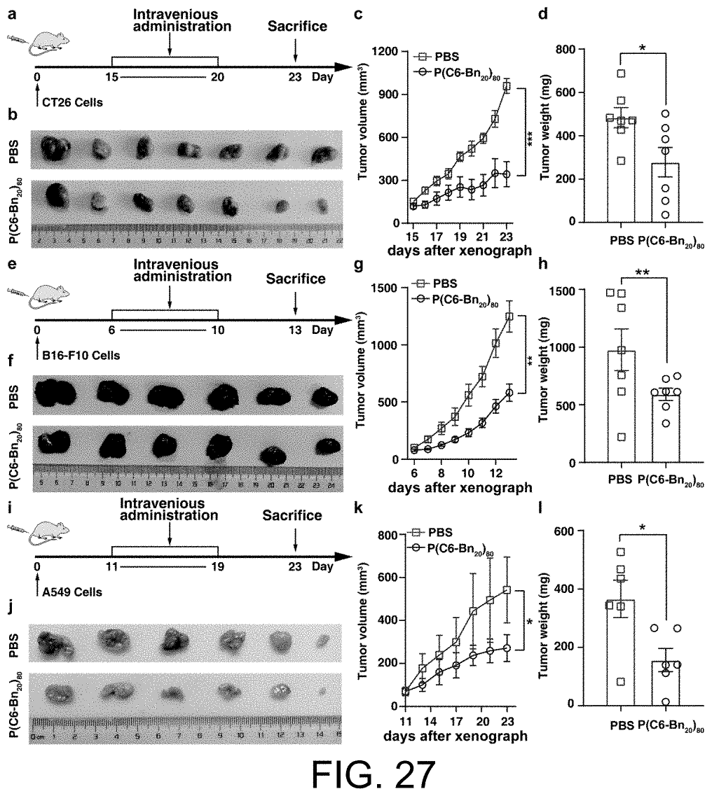

Subcutaneous injecting on the back of female mice (C57BL/6, 6-8 weeks) with 1×10⁶/mL Panc02 cells suspension (100 gL) to establish the subcutaneous tumor model of pancreatic cancer; subcutaneous injecting on the back of female mice (C57BL/6, 6-8 weeks) with 1×10⁶/mL B16-F10 cells suspension (100 μL) to establish melanoma subcutaneous tumor model; subcutaneous injecting on the back of female mice (BALb/c, 6-8 weeks) with 5×10⁵ mL CT26 cells suspension (100 μL) to establish the subcutaneous tumor model of colorectal cancer; subcutaneous injecting on the back of female mice (Nu^{-/-}Nu^{-/-}, 6-8 weeks) with 5×10⁶/mL A549 cells suspension (100μL) to establish the subcutaneous tumor model of lung cancer. When the tumor grew to 50-100 mm³ (tumor volume=length×wide×Width/2), the mice were divided into groups and administered via tail vein. Negative control group was set up through injecting only PBS solution of equal volume. The tumor size was measured with vernier caliper, and the weight of mice was recorded. The anti-tumor effect of Panc02 model in vivo is shown in FIG. 26. According to the tumor growth curve, compared to the PBS group, the tumor volume of three treatment groups (treated with P (C6-Bn₂₀)₈₀ nanoparticles material) was inhibited to a certain extent, and the growth of tumor volume of the 50 mg/kg treatment group was significantly slowed down. Meanwhile, after the whole treatment cycle, there were no difference in the weight of mice between the experimental groups and the PBS control group, indicating the treatment process would not cause weight loss of mice. Through tumor weighting in vitro, it can be found that the weight of tumor of the administration groups with different doses were lighter than that of the PBS control group, among which the weight of the administration group with a dose of 50 mg/kg was the lightest. The tumor weight in vitro was consistent with the tumor growth curve. In other tumor models (dosage was 50 mg/kg), P (C6-Bn₂₀)₈₀ material had good inhibition effect on various tumor models (FIG. 27).

### Embodiment 7: Killing Effect of Polymer Materials on Bacteria at Different pH.

The embodiment studied the effect of the polymer materials of the present disclosure on killing bacteria under acidic conditions, and experiments showed that the polymer materials of the present disclosure could effectively kill bacteria under infected acidity.

Monoclonal colonies of bacteria (Escherichia coli ATCC25922, Salmonella ATCC 14028, Staphylococcus aureus ATCC6538, Klebsiella pneumoniae ATCC700603, Pseudomonas aeruginosa ATCC27853, Enterococcus faecalis ATCC29212) were selected and cultured in 1 mL LB broth by shaking at 37° C. (220 rpm) overnight. Then 5 μL of bacterial suspension was inoculated into 1 mL fresh LB broth and shaken at 37° C. overnight. The bacterial suspension was centrifuged at 10000 rpm for 1 min, and the supernatant was decanted. Then 1 mL PBS was added for resuspension cleaning and centrifugation again. After repeated cleaning for three times, cells were resuspended with PBS.

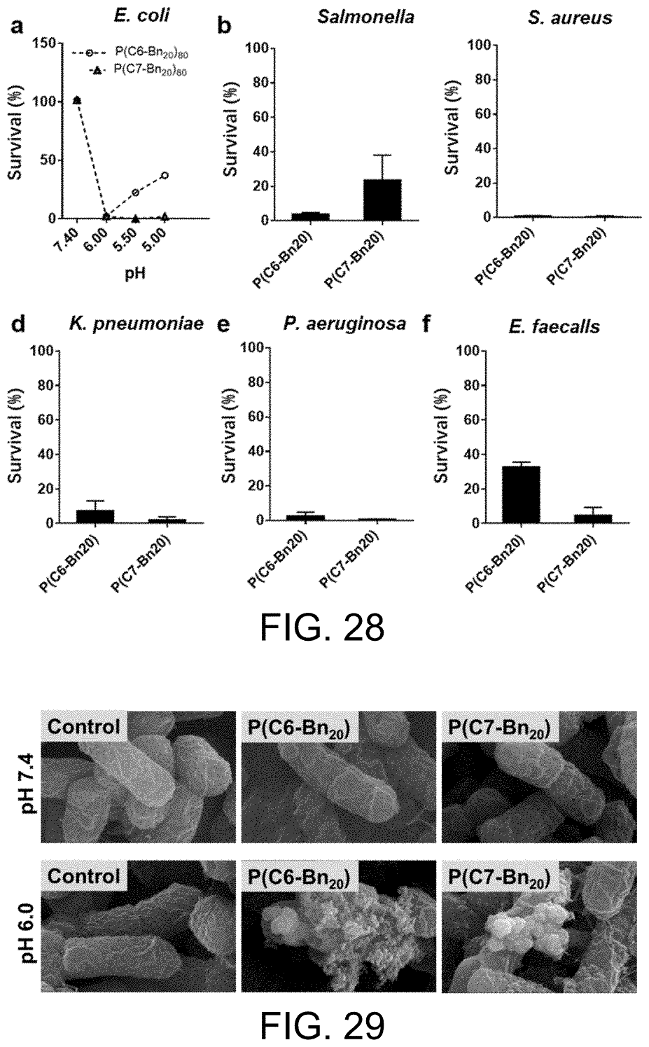

The bacterial solution of Escherichia coli vas dilutetod to 1×10⁶ CFU/mL with M9 medium at pH 5.00, 5.50, 6.00 and 7.40, respectively, and then mixed with equal volume of 32 μg/mL and 0 μg/mL polymer material solution (prepared with M9 medium with corresponding pH of 5.00, 5.50, 6.00 and 7.40 respectively). After incubation at 37° C. for 1 hour, the bactericidal performance was evaluated by dilution plate colony count method. The sample without polymer material solution was taken as the control group. In additon, control groups were set corresponding to each pH. The bacterial survival rate was calculated by Formula (1). The results are shown in FIG. 28a.

$$Survial ( \% ) = \frac{{N}_{Sample}}{{N}_{Control}} \times 100 \qquad Formula ( 1 )$$

Wherein, the samples without polymer material solution were taken as the control group. N_{Sample} and N_{Control} were the number of viable colonies (CFU) in the experimental group and the control group at the same dilution ratio. The result is the average of the parallel samples, wherein n=3.

The polymer materials used were: P (C6-Bn₂₀)₈₀ nanoparticles and P (C7-Bn₂₀)₈₀ nanoparticles. As shown in FIG. 28a, the killing of P (C6-Bn₂₀)₈₀ nanoparticles and P (C7-Bn₂₀)₈₀ nanoparticles to Escherichia coli was pH dependent. They had no obvious killing effect on bacteria at pH 7.40, but showed high killing activity under acidic conditions (pH 5.00, 5.50, 6.00). Using the same experimental method, it was found that P (C6-Bn₂₀)₈₀ nanoparticles and P (C7-Bn₂₀)₈₀ nanoparticles had highly effective killing effects on Salmonella, Staphylococcus aureus, Klebsiella pneumoniae, Pseudomonas aeruginosa, Enterococcus faecalis, etc. at a concentration of 32 μg/mL and at pH 6.00. The results are shown in FIG. 28b-f.

Furthermore, the monoclonal colonies of Escherichia coli (AFCC 25922) were selected and added into 1 mL LB broth, and cultured overnight by shaking at 37° C. (220 rpm). Then 200 μL of bacterial suspension was inoculated into 20 mL of fresh LB broth, and was shaken at 37° C. for 3 hours. The bacterial suspension was centrifuged at 40000 rpm for 10 min, and the supernatant was decanted. Then 1 mL PBS was added for resuspension cleaning and centrifugation again. After repeated cleaning for three times, the cells were resuspended with PBS.

The bacterial solution was dilutetod to 2×10⁹ CFU/mL with M9 medium at pH of 6.00 and 7.40, respectively, and then mixed with the equal volume of 256 μg/mL and 0 μg/mL polymer material solution (prepared with M9 medium with corresponding pH of 6.00 and 7.40 respectively). After incubation at 37° C. for 1 hour, electron microscope fixative was added and fixed overnight. Then PBS was used to wash them for three times, and each incubation was 20 minutes. Ethanol with gradient concentration (30%, 50%, 70?, 90%, 95%) was used to dehydrate cells in a gradient manner for 15 minutes each time. Then the bacterial solution was incubated with anhydrous ethanol for 3 times, and each incubation was 15 minutes; finally, the bacterial solution was incubated twice with tert butyl alcohol, and each incubation was 20 minutes. After freeze-drying, the samples were sprayed with gold and observed by field emission scanning electron microscope. The results were shown in FIG. 29 P(C6-Bn₂₀)₈₀ nanoparticles and P (C7-Bn₂₀)₈₀ nanoparticles had no obvious effect on the morphology of Escherichia coli at pH 7.4. However, at pH 6.0, after treatment with P(C6-Bn₂₀)₈₀ nanoparticles and P (C7-Bn₂₀)₈₀ nanoparticles, there were obvious holes on the bacterial cell membrane, indicating that P(C6-Bn₂₀)₈₀ nanoparticles and P (C7-Bn₂₀)₈₀ nanoparticles could kill bacteria by destroying the bacterial cell membrane under acidic conditions.

The technical features of the embodiments above can be combined arbitrarily. To simplify the description, all possible combinations of technical features of the embodiments above are not described. However, as long as there is no contradiction in the combination of these technical features, they should be considered to be the scope recorded in the description.

The embodiments above express several implementations of the present disclosure only, The description of the embodiments is relatively specific and detailed, but may not therefore be construed as limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. A polymer material, having the structure shown in Formula (I) 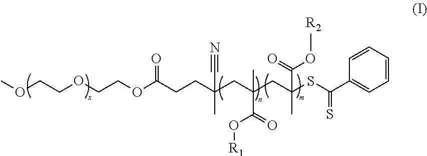
wherein R₁ is selected from —R₃—N(R₄R₅);
₂ is selected from: alkyl, aryl, aryl substituted alkyl;
R₃ is selected from: alkylene;
R₄ and R₅ are independently selected from alkyl groups, or R₄ and R₅ together with the attached nitrogen atom form a heterocyclic alkyl group;
x is greater than 0;
n+m is not less than 20.

2. The polymer material according to claim 1, wherein R₂ is selected from: C₁-C₁₅ alkyl, C₆-C₁₄ aryl, C₆-C₁₄ aryl substituted C₁-C₁₅ alkyl.

3. The polymer material according to claim 2, wherein R₂ is selected from: C₁-C₁₂ alkyl, phenyl, naphthyl, phenyl-substituted C₁-C₃ alkyl, naphthyl-substituted C₁-C₃ alkyl alkyl.

4. The polymer material according to claim 3, wherein R₂ is selected from: methyl, ethyl, propyl, isopropyl, butyl, pentyl, hexyl, heptyl, isooctyl, nonyl, decyl, undecyl, dodecyl, tetradecyl, phenyl, naphthyl, benzyl, naphthalene methyl.

5. The polymer material according to claim 1, wherein R₃ is selected from: C₁-C₆ alkylene.

6. The polymer material according to claim 5, wherein R₃ is selected from: methylene, ethylidene, propylidene.

7. The polymer material according to claim 1, wherein R₄ and R₅ are independently selected from C₁-C₆ alkyl, or R₄ and R₅ together with the attached nitrogen atom form a 5-10 membered heterocyclic alkyl.

8. The polymer material according to claim 7, wherein R₄ and R₅ are independently selected from C₁-C₄ alkyl, or R₄ and R₅ together with the attached nitrogen atom form a 5-8 membered heterocyclic alkyl.

9. The polymer material according to claim 8, wherein R₄ and R₅ together with the attached nitrogen atom form the following groups:

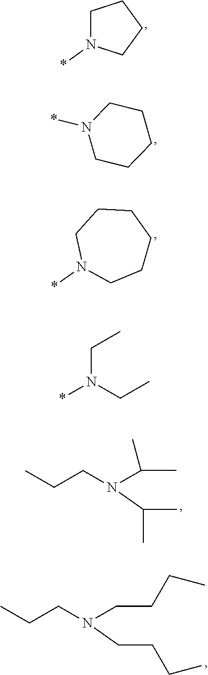

10. The polymer material according to claim 1, having the structure shown in Formula (II): 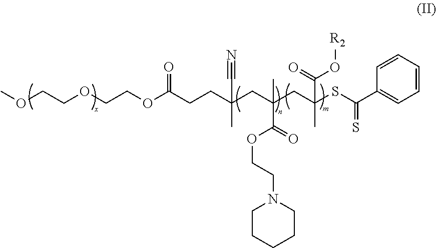
wherein R₂ is selected from: methyl, ethyl, propyl, isopropyl, butyl, penyl, hexyl, heptyl, isooctyl, nonyl, decyl, undecyl, dodecyl, tetradecyl, phenyl, naphthyl, benzyl, naphthalene methyl.

11. The polymer material according to claim 1, having the structure shown in Formula (III): 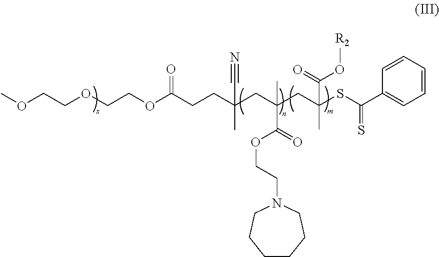
wherein R₂ is selected from: methyl, ethyl, propyl, isopropyl, butyl, penyl, hexyl, heptyl, isooctyl, nonyl, decyl, undecyl, dodecyl, tetradecyl, phenyl, naphthyl, benzyl, naphthalene methyl.

12. The polymer material according to claim 1, having the structure shown in Formula (IV): 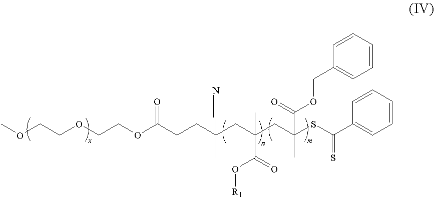
wherein R₁ is selected from:

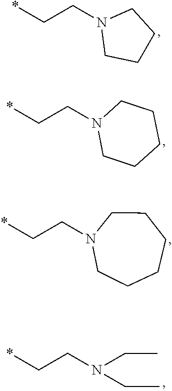

13. The polymer material according to claim 1, Therein n+m is not less than 70.

14. The polymer material according to claim 13, wherein n+m is 75-200.

15. The polymer material according to claim 1, wherein m is 5%-50% of n+m.

16. The polymer material according to claim 15, wherein m is 18%-30% of n+m.

17. The polymer material according to claim 16, wherein in is 20%-25% of n+m.

18. The polymer material according to claim 1, wherein x is 10-250.

19. The polymer material according to claim 1, having the structure shown in Formula (V) or Formula (VI):

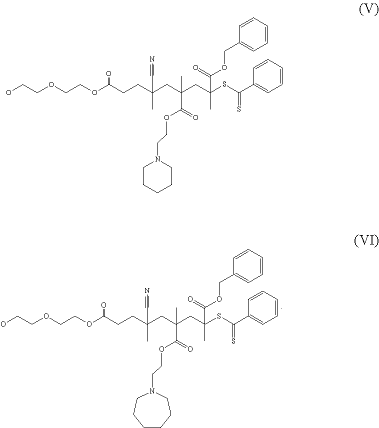

20. A nanoparticle of the polymer material, being formed by self-assembly of the polymer material according to claim 1 in an aqueous medium.

21. A preparation method of the nanoparticle of polymer material according to claim 20, comprising the following steps: dissolving the polymer material in dimethylformamide, and then adding the obtained solution into deionized water in drops under stirring, continuing stirring, removing the solvent through dialysis to obtain the nanoparticles of the polymer material.

22. The preparation method of the nanoparticle of the polymer material according to claim 21, comprising the following steps: dissolving the polymer material in dimethylformamide at a ratio of 45-55 mg: 1 mL, and then adding the obtained solution into deionized water in drops under stirring at a speed of 1200-1700 rpm, continuing to stir at a speed of 800-1200 rpm for 8-12 minutes, removing the solvent by dialysis using a dialysis bag with a cutoff molecular of 10000˜20000, to obtain the nanoparticle of the polymer material.

23. A drug for preventing and/or treating tumors or treating bacterial infections, prepared from active ingredients and pharmaceutically acceptable excipients, wherein the active ingredients comprise the polymer material according to claim 1.

24. The drug according to claim 23, wherein the tumors are pancreatic cancer, melanoma, colorectal cancer, lung cancer, tongue squamous cell cancer, cervical cancer, ovarian cancer, osteosarcoma, liver cancer, breast cancer, bladder cancer, epithelial ovarian cancer.

25. The drug according to claim 23, wherein the bacteria are gram-negative bacteria, gram-negative pseudomonas, gram-positive staphylococcus, gram-positive coccus, gram-positive streptococcus.

26. The drug according to claim 23, wherein the bacteria are Escherichia coli, Salmonella, Staphylococcus aureus, Klebsiella pneumoniae, Pseudomonas aeruginosa, Enterococcus faecalis, Streptococcus pyogenes, Streptococcus pneumoniae, Acinetobacter baumannii, Diplococcus pneumoniae, Pseudomonas aeruginosa.
